(19) United States
(12) Patent Application Publication
(10) Pub. No.: US 2009/0118255 A1
(43) Pub. Date: May 7, 2009

(54) Crp Lowering Agent

(75) Inventors: Yoshimi Imura, Osaka-shi (JP); Ryuichi Tozawa, Osaka-shi (JP); Tomoyuki Nishimoto, Osaka-shi (JP)

Correspondence Address: EDWARDS ANGELL PALMER & DODGE LLP, P.O. BOX 55874, BOSTON, MA, 02205, US

(73) Assignee: TAKEDA PHARMACEUTICAL COMPANY LIMITED, Osaka (JP)

(21) Appl. No.: 11/659,941

(22) PCT Filed: Aug. 8, 2005
(86) PCT No.: PCT/JP05/14863

§ 371 Date: Feb. 9, 2007

(30) Foreign Application Priority Data
Aug. 9, 2004 (JP) 2004-232605

(51) Int. Cl. A61K 31/553; C07D 413/02

(52) U.S. Cl. 514/211.05; 540/490

(57) ABSTRACT

The present invention provides a novel drug which is useful as a preventive and/or therapeutic agent for various diseases involved in elevation of CRP level, in particular, inflammatory disease and cancer comprising a compound having inhibitory activity against squalene synthase or a salt thereof, or a prodrug thereof.

### TECHNICAL FIELD

The present invention relates to a CRP lowering agent comprising a compound having a squalene synthase inhibitory activity or a salt thereof, or a prodrug thereof.

### BACKGROUND ART

C-reactive protein (CRP) is a protein which binds to C-polysaccharide of Diplococcus pneumoniae, and it is known that its blood level is increased due to inflammatory diseases (infection, rheumatism, etc.). Recently, it has been revealed that CRP is not only a marker for inflammatory diseases as conventionally known but also a factor which directly exaggerate inflammatory diseases. For example, CRP is shown to increase the expression of matrix metalloprotease (MMP)-1 of human macrophage and enhance collagenase activity (Takeyla N. Williams et al, Arteriosclerosis Thrombosis Vascular Biology, 2004, Vol. 24, pp. 61-66). It is suggested that CRP is involved in the destabilization of plaque, since complications such as acute coronary syndrome (ACS) in arteriosclerosis (atherosclerosis) is considered to be developed by rendering the collagen fiber constituting fibrous coat of plaque vulnerable by MMPs secreted from foam cells, followed by plaque rupture. In addition, it has been reported that CRP increases the expression of PAI-1 in human arterial endothelial cell (Sridevi Devaraj et al., Circulation, 2003, Vol. 107, pp. 398-404). When an inflammation and thrombus are increased due to the increase in expression of MMP and PAI-1, functional disorders of blood vessel endothelial cell are caused, and inflammatory cells such as monocyte and macrophage penetrate into blood vessel. Activated macrophages accumulate oxidation-denatured LDL cholesterols to turn into foam cells, and the arteriosclerotic (atherosclerotic) lesion is further progressed by generating and releasing inflammatory cytokines and growth factors. Thus, it is suggested that CRP is involved in the progress of arteriosclerotic (atherosclerotic) plaque.

Furthermore, inhibitors of MMP-1 have been developed worldwide as treating drugs for rheumatism and cancer, and CRP lowering agents are expected to be useful as treating drugs for rheumatism and cancer since the CRP lowering agents inhibit the increase in the expression of MMP-1. Similarly, PAI-1 inhibitors are under development in the world as an antithrombotic drug, and CRP lowering agents are supposed to be useful as antithrombotic drug since the CRP lowering agents inhibit the increase in the expression of PAI-1. That CRP is a risk factor for thrombus formation is more directly indicated by the fact that in a transgenic mouse enriched with CRP gene, the frequency in which thrombogenic occlusion is formed after femoral artery disorder is remarkably higher compared to that of wild mouse (Haim D. Danenberg et al., Circulation, 2003, Vol. 108, pp. 512-515).

It is known that the compound having a squalene synthase inhibitory activity is useful as a preventive and therapeutic agent for hyperlipidemia, arteriosclerosis (atherosclerosis) and the like, triglyceride lowering agents, hypolipidemic agents, high density lipoprotein-cholesterol elevating agent, antimycotic agent, skeletal muscle protecting agent, and the like (JP-A Nos. 6-239843, 8-157369, 9-136880, 2002-080468, and 2002-205956), however there has not been ever reported that a CRP lowering action is observed either in vitro or in vivo. In addition, it is known that in cholesterol lowering agent, HMG-CoA reductase inhibitor lowers CRP, but it is also known that ezetimibe which is another type of cholesterol lowering agent has no CRP lowering activity (Christie M. Ballantyne et al., Circulation, 2003, Vol. 107, pp. 2409-2415; Philip T. Sager et al., American Journal of cardiology, 2003, Vol. 92, pp. 1414-1418). Therefore, cholesterol lowering agents do not always have a CRP lowering activity.

### DISCLOSURE OF INVENTION

As described above, since it is suggested that CRP takes a positive role in the occurrence/progression of inflammatory diseases including arteriosclerosis (atherosclerosis), a drug having CRP lowering activity is expected to be useful for the prevention and treatment of these diseases. However, existing drugs having CRP lowering activity have a serious side effect in some cases (for example, HMG-CoA reductase inhibitor causes rhabdomyolysis), therefore the development of clinically more safe and useful new drug is presently awaited.

In view of the above circumstances, the present inventors have intensively investigated and as a result, unexpectedly, it has been found first that a compound having a squalene synthase inhibitory activity is clinically useful as a medicine for lowering CRP, and thus the present invention is completed.

That is, the present invention relates to:

(1) a CRP lowering agent comprising a compound having a squalene synthase inhibitory activity or a salt thereof, or a prodrug thereof;

(2) the agent according to the above-mentioned (1) which is a preventive and/or therapeutic agent for inflammatory diseases;

(3) the agent according to the above-mentioned (1) which is a preventive and/or therapeutic agent for hyper C-reactive proteinemia (hereinafter referred to as hyper CRPemia in some occasions);

(4) the agent according to the above-mentioned (1) which is an inhibitor of development of arteriosclerotic (atherosclerotic) plaque or a stabilizer thereof;

(5) the agent according to the above-mentioned (1),

wherein the compound having a squalene synthase inhibitory activity is a compound represented by the formula:

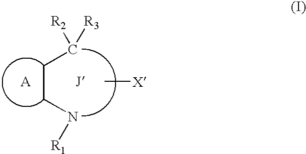

wherein, R₁ represents a hydrogen atom or an optionally substituted hydrocarbon group, R₂ and R₃ are the same or different and represent a hydrogen atom, an optionally substituted hydrocarbon group or an optionally substituted heterocyclic group, X′ represents a group comprising an optionally esterified carboxyl group, an optionally substituted carbamoyl group, an optionally substituted hydroxy group, an optionally substituted amino group or an optionally substituted heterocyclic residue having a hydrogen atom which can be deprotonated, Ring A represents an optionally substituted benzene ring or an optionally substituted heterocyclic ring, Ring J′ represents a 7- to 8-membered heterocyclic ring containing 3 or less hetero atoms as ring constituent atoms, and Ring J′ may further have a substituent in addition to R₁, R₂, R₃, and X′;

(6) the agent according to the above-mentioned (1), wherein the compound having a squalene synthase inhibitory activity is a compound represented by the formula:

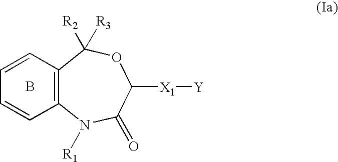

wherein, R₁ represents a hydrogen atom or an optionally substituted hydrocarbon group, R₂ and R₃ are the same or different and represent a hydrogen atom, an optionally substituted hydrocarbon group or an optionally substituted heterocyclic group, X₁ represents a bond or a divalent atomic chain, Y represents an optionally esterified carboxyl group, an optionally substituted carbamoyl group, an optionally substituted hydroxy group, an optionally substituted amino group, or an optionally substituted heterocyclic residue having a hydrogen atom which can be deprotonated, Ring B represents an optionally substituted benzene ring;

(7) the agent according to the above-mentioned (1), wherein the compound having a squalene synthase inhibitory activity is a compound represented by the formula:

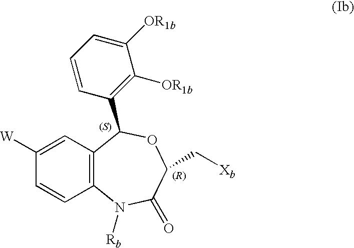

wherein, R_{b} represents a lower alkyl group optionally substituted with an optionally substituted hydroxy group, X_{b} represents an optionally substituted carbamoyl group or an optionally substituted heterocyclic group having a hydrogen atom which can be deprotonated, R_{1b} represents a lower alkyl, and W represents a halogen atom;

(8) the agent according to the above-mentioned (7), wherein R_{b} is a C₁₋₆ alkyl which may have 1 to 3 substituents selected from hydroxy group, acetyloxy, propionyloxy, t-butoxycarbonyloxy, palmitoyloxy, dimethylaminoacetyloxy and 2-aminopropionyloxy;

(9) the agent according to the above-mentioned (7), wherein R_{1b} is methyl;

(10) the agent according to the above-mentioned (7), wherein W is chlorine atom;

(11) the agent according to the above-mentioned (7), wherein X_{b} is a group represented by the formula:

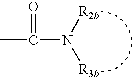

wherein R_{2b} and R_{3b} are each a hydrogen atom, an optionally substituted hydrocarbon group, an optionally substituted heterocyclic group or an acyl group, or R_{2b} and R_{3b} may be combined together with the adjacent nitrogen atom to form an optionally substituted 5- or 6-membered nitrogen-containing heterocyclic ring which may contain 1 to 3 hetero atoms selected from a nitrogen atom, a sulfur atom and an oxygen atom as ring constituent atoms;

(12) the agent according to the above-mentioned (7), wherein X_{b} is a group represented by the formula:

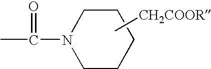

wherein R″ represents hydrogen atom or a C₁₋₄ alkyl;

(13) the agent according to the above-mentioned (1), wherein the compound having a squalene synthase inhibitory activity is N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-yl]acetyl]piperidine-4-acetic acid or N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepine-3-yl]acetyl]piperidine-4-acetic acid;

(14) a method for preventing and/or treating a disease involved in elevation of CRP, which comprises inhibiting a squalene synthase in mammals;

(15) a method for preventing and/or treating a disease involved in elevation of CRP, which comprises administering an effective amount of a compound having a squalene synthase inhibitory activity, or a prodrug thereof, or a salt thereof to a mammal; and

(16) use of a compound having a squalene synthase inhibitory activity, or a prodrug thereof, or a salt thereof for producing a preventive and/or therapeutic agent of a disease involved in elevation of CRP.

### BEST MODE FOR CARRYING OUT THE INVENTION

As the “compound having a squalene synthase inhibitory activity” to be used in the present invention, any compound can be used as long as it has a squalene synthase inhibitory activity, for example, squalenestatins (e.g., U.S. Pat. Nos. 5,506,262, 5,430,055, 5,409,950, 5,369,125, JP-A Nos. 7-173166, 9-124655, 9-227566, “Annual Review of Microbiology”, Vol. 49, pp. 607-639, 1995, “Journal of Medicinal Chemistry”, Vol. 38, pp. 3502-3513, 1995, “Journal of Medicinal Chemistry”, Vol. 39, pp. 207-216, 1996, “Journal of Medicinal Chemistry”, Vol. 39, pp. 1413-1422, 1996, etc.), a phosphate compound and a carboxylic acid compound of a substrate analog (e.g., U.S. Pat. Nos. 5,374,628, 5,441,946, 5,428,028, JP-A No. 7-041554, WO95/04025, “Journal of Medicinal Chemistry”, Vol. 38, pp. 2596-2605, 1995, “Arzniemittel-Forschung Drug Research”, Vol. 46, pp. 759-762, 1996, “Journal of Medicinal Chemistry”, Vol. 31, pp. 1869-1871, 1988, “Journal of Medicinal Chemistry”, Vol. 39, pp. 657-660, 1996, “Journal of Medicinal Chemistry”, Vol. 39, pp. 661-664, 1996), carboxylic acid derivatives (e.g., WO97/40006, WO96/33159, WO95/21834, WO97/48701, EP-A Nos. 645377, 645378, 814080, 790235, JP-A Nos. 7-173120, 10-316634, 10-298134, 10-298177, 10-316617, 9-136880, WO2000/00458, WO2001/98282, WO98/29380, “Bioorganic Medicinal Chemistry Letters”, Vol. 5, pp. 1989-1994, 1995, “Bioorganic Medicinal Chemistry Letters”, Vol. 6, pp. 463-466, 1996, “Journal of Medicinal Chemistry”, Vol. 40, pp. 2123-2125, 1997, etc.), an amine-based compound such as quinuclidine derivatives (e.g., U.S. Pat. Nos. 5,385,912, 5,494,918, 5,395,846, 5,451,596, JP-A Nos. 8-134067, 2000-169474, 10-152453, 2000-502716, WO94/03541, WO 94/05660, WO95/35295, WO96/26938, WO95/31458, WO95/00146, WO97/25043, WO98/12170, etc.), and Zaragozic acids, particularly, a compound represented by the formula:

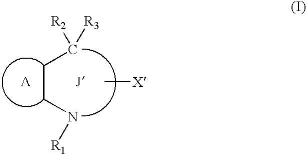

wherein, R₁ is a hydrogen atom or an optionally substituted hydrocarbon group, R₂ and R₃ are the same or different and a hydrogen atom, optionally substituted hydrocarbon group or an optionally substituted heterocyclic group, X′ is a group comprising an optionally esterified carboxyl group, an optionally substituted carbamoyl group, an optionally substituted hydroxy group, an optionally substituted amino group or an optionally substituted heterocyclic residue having a hydrogen atom which can be deprotonated, Ring A is an optionally substituted benzene ring or an optionally substituted heterocyclic ring, Ring J′ is a 7- to 8-membered heterocyclic ring containing 3 or less hetero atoms as ring constituent atoms, and Ring J′ may further have a substituent in addition to R₁, R₂, R₃, and X′; or a compound represented by the formula:

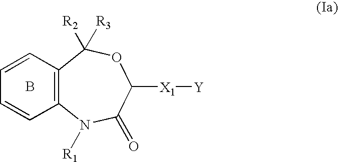

wherein, R₁ is a hydrogen atom or an optionally substituted hydrocarbon group, R₂ and R₃ are the same or different and a hydrogen atom, optionally substituted hydrocarbon group or optionally substituted heterocyclic group, X₁ is a bond or divalent atomic chain, Y is an optionally esterified carboxyl group, an optionally substituted carbamoyl group, an optionally substituted hydroxy group, an optionally substituted amino group or an optionally substituted heterocyclic residue having a hydrogen atom which can be deprotonated, Ring B is an optionally substituted benzene ring; or the like is preferably used.

Examples of other squalene synthase inhibitors include A-104109 (Abbott Laboratories),

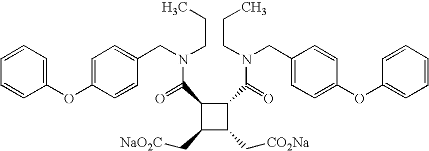

F-10863-A (Zaragozic acid D3, Sankyo Co., Ltd.), bisphosphonic acid derivatives such as ER-28448, ER-27856 (ER-28448 prodrug), and quinuclidine derivatives (Eisai) such as ER-119884 and ER-132781,

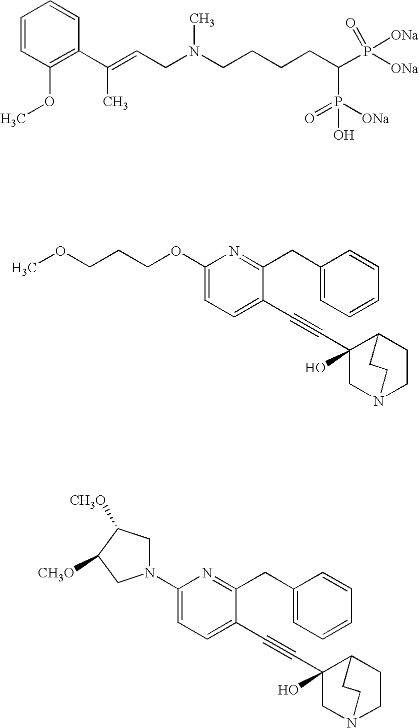

### RPR-107393 and RPR-101821 (Aventis Pharma Ltd.),

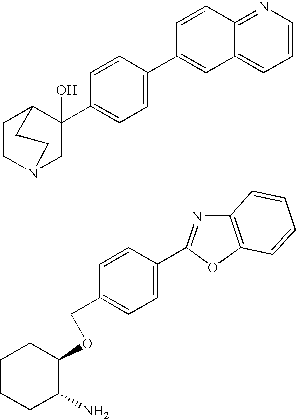

thiadiazole derivatives (NovoNordisk),

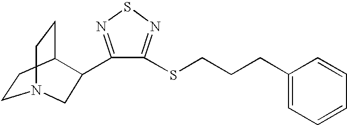

isopropylamine derivatives and quinuclidine derivatives (Yamanouchi Pharmaceutical Co., Ltd.),

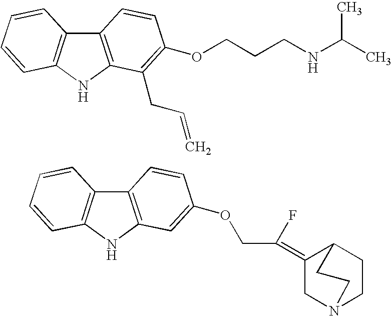

isoquinuclidine derivatives (Kotobuki pharmaceutical Co., Ltd.)

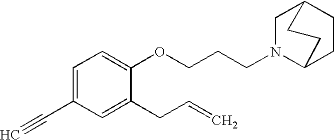

malonic acid derivatives (Nippon Kayaku Co., Ltd.),

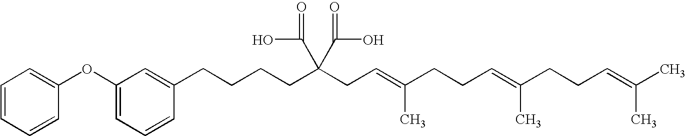

propionyl derivatives (Daiichi Pharmaceutical Co., Ltd.)

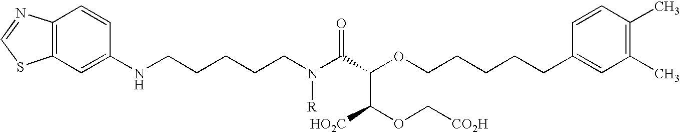

wherein R is hydrogen atom or methyl group, SQ-34919, SQ-32709, BMS-187745 and BMS-188494 (Bristol-Myers Squibb Company)

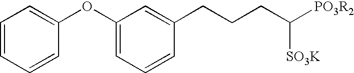

wherein R is potassium atom or —CH₂OCOC(CH₃)₃, J-104118 (Merck & Co., Inc.)

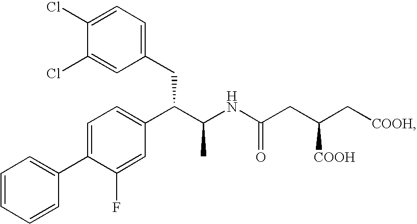

quinuclidine derivatives (AstraZeneca)

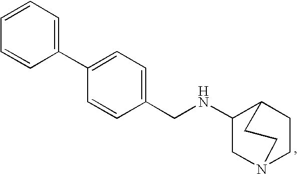

### SDZ-266-806 (Novartis Pharma)

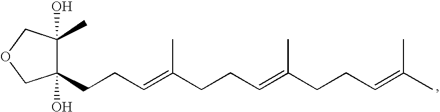

tricyclic fused ring derivatives disclosed in WO 98/12170:

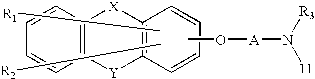

wherein, R₁ and R₂ are the same or different and each represents hydrogen atom, or an optionally substituted lower alkyl group or lower alkenyl group, X and Y are the same or different and each represents a bond, or a group represented by —CH₂—, —CO—, —O— or —NR₄—, A represents an alkylene group or an alkenylene group, R₃ represents a lower alkyl group, a cycloalkyl group or a lower alkylaryl group, and R₄ represents hydrogen atom or —CO-lower alkyl group,
quinuclidine compounds disclosed in WO 01/23383:

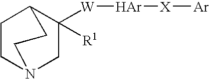

wherein, R₁ represents (1) hydrogen atom or (2) hydroxy group, HAr represents an aromatic heterocyclic ring which may be substituted with 1 to 3 groups, Ar represents an optionally substituted aromatic ring, W represents a chain represented by (1) optionally substituted —CH₂—CH₂—, (2) optionally substituted —CH═CH—, (3) —C≡C—, (4) —NH—CO—, (5) —CO—NH—, (6) —NH—CH₂—, (7) —CH₂—NH—, (8) —CH₂—CO—, (9) —CO—CH₂—, (10) —NH—S(O)₁—, (11) —S(O)₁—NH—, (12) —CH₂—S(°)₁—, or (13) —S(O)ₙ—CH₂— (1 represents 0, 1 or 2), and X represents a chain represented by (1) single bond, (2) optionally substituted C₁₋₆ alkylene chain, (3) optionally substituted C₂₋₆ alkenylene chain, (4) optionally substituted C₂₋₆ alkynylene chain, (5) formula -Q- (wherein, Q represents oxygen atom, sulfur atom, CO or N(R²) (wherein, R² represents C₁₋₆ alkyl group or C₁₋₆ alkoxy group)), (6) —NH—CO—, (7) —CO—NH—, (8) —NH—CH₂—, (9) —CH₂—NH—, (10) —CH₂—CO—, (11) —CO—CH₂—, (12) —NH—S(O)ₘ—, (13) —S(O)ₘ—NH—, (14) —CH₂—S(O)ₘ—, (15) —S(O)ₘ—CH₂— (wherein, m represents 0, 1 or 2), or (16) —(CH₂)ₙ—O— (wherein, n represents an integer of 1 to 6), and
pyrrolidine derivatives disclosed in WO 02/083636:

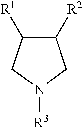

wherein, R¹ and R² represent independently a halogen atom, hydroxy group, or a group represented by formula —O—R₁₀ (wherein, R₁₀ means a C₁₋₆ alkyl group which may have 1 to 3 substituents selected from substituent group A consisting of a halogen atom, hydroxy group, methoxy group, phenyl group, C₃₋₈ cycloalkyl group and C₁₋₆ alkoxy group, or a C₃₋₈ cycloalkyl group which may have 1 to 3 substituents selected from the above-mentioned substituent group A), respectively, R³ represent benzyl group which may have 1 to 3 of methoxy group or nitro group as substituents, or hydrogen atom; provided that (1) the case where R¹ and R² are the same and (2) the case where one of R¹ and R² is hydroxy group and the other is ethoxy group or chlorine atom, are excluded,
and such squalene synthase inhibitors can be also used in an agent of the present invention.

The “compound having squalene synthase inhibitory activity” used in the present invention can be used in a form of a salt or a prodrug.

As for a “salt” of the compound having squalene synthase inhibitory activity used in the present invention, a pharmaceutically acceptable salt or a physiologically acceptable acid addition salt is preferred. For such salts, for example, inorganic acids (e.g., hydrochloric acid, phosphoric acid, hydrobromic acid, sulfuric acid, etc.) or organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid, etc.) or the like are used. Further, in the case that the “compound having squalene synthase inhibitory activity” used in the present invention has an acidic group such as carboxylic acid or the like, the “compound having squalene synthase inhibitory activity” may form salts with, for example, an inorganic base (e.g., an alkali metal or alkaline earth metal such as sodium, potassium, calcium, magnesium, or ammonia, etc.) or an organic base (e.g., tri-C₁₋₃ alkylamine such as triethylamine, etc.).

The “prodrug” of the compound having squalene synthase inhibitory activity [hereinafter, referred to as “SSI Compound”] used in the present invention or a salt thereof refers to a compound which is converted to the SSI Compound by a reaction in vivo under the physiological condition with an enzyme, a gastric acid or the like, that is, a compound which is converted to the SSI Compound by enzymatic oxidation, reduction, hydrolysis, etc.; a compound which is converted to the SSI Compound by hydrolysis or the like with gastric acid, etc.; or the like. Examples of the prodrug of the SSI Compound include a compound wherein an amino group of the SSI Compound is acylated, alkylated or phosphorylated (e.g., a compound wherein an amino group of the SSI Compound is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, etc.); a compound wherein a hydroxy group of the SSI Compound is acylated, alkylated, phosphorylated or borylated (e.g., a compound wherein a hydroxy group of the SSI Compound is acetylated, palmitoylated, propanoylated, pivaloylated, succinylated, fumarylated, alanylated or dimethylaminomethylcarbonylated, etc.); or a compound wherein a carboxyl group of the SSI Compound is esterified or amidated (e.g., a compound wherein a carboxyl group of the SSI Compound is ethylesterified, phenylesterified, carboxymethylesterified, dimethylaminomethylesterified, pivaloyloxymethylesterified, ethoxycarbonyloxyethylesterified, phthalidylesterified, (5-methyl-2-oxo-1,3-dioxolen-4-yl)methylesterified, cyclohexyloxycarbonylethylesterified or methylamidated, etc.); and the like. These compounds can be prepared from the SSI Compound by a per se known method.

In addition, the prodrug of the SSI Compound may be a compound which is converted into the SSI Compound under the physiological conditions as described in “Pharmaceutical Research and Development”, Vol. 7 (Molecular Design), pp. 163-198, published in 1990 by Hirokawa Publishing Co.

Further, the SSI Compound may be hydrated.

When the optically active form of the SSI Compound is needed, it can be obtained, for example, by using an optically active starting material, or by using a conventional method to optically resolve the racemic form of the SSI Compound. Further, when the SSI Compound contains an asymmetric carbon in its molecule and has two stereoisomers of R-configuration and S-configuration, any isomer or a mixture thereof is included within the scope of the present invention.

In the formulae (I) and (Ia), examples of the hydrocarbon group in the “optionally substituted hydrocarbon group” represented by R₁ include an aliphatic chain (acyclic) hydrocarbon group, an alicyclic hydrocarbon group and an aryl group, and among these, aliphatic chain hydrocarbon group is preferred.

The aliphatic chain hydrocarbon group of the hydrocarbon group includes a linear or branched aliphatic hydrocarbon group such as an alkyl group, an alkenyl group, and an alkynyl group. Among these, the branched alkyl group is preferred. Examples of the alkyl group include C₁₋₇ alkyl such as methyl, ethyl, n-propyl, isopropyl, n-butyl, isobutyl, sec-butyl, tert-butyl, n-pentyl, isopentyl, neopentyl, 1-methylpropyl, n-hexyl, isohexyl, 1,1-dimethylbutyl, 2,2-dimethylbutyl, 3,3-dimethylbutyl, 3,3-dimethylpropyl, 2-ethylbutyl, n-heptyl and the like. Inter alia, C₃₋₅ alkyl such as n-propyl, isopropyl, isobutyl, neopentyl and the like is preferred, and isobutyl, neopentyl and the like are particularly preferred.

Examples of the alkenyl group include C₂₋₆ alkenyl such as vinyl, allyl, isopropenyl, 2-methylallyl, 1-propenyl, 2-methyl-1-propenyl, 2-methyl-2-propenyl, 1-butenyl, 2-butenyl, 3-butenyl, 2-ethyl-1-butenyl, 2-methyl-2-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl and the like. Inter alia, vinyl, allyl, isopropenyl, 2-methylallyl, 2-methyl-1-propenyl, 2-methyl-2-propenyl, 3-methyl-2-butenyl and the like are particularly preferred. Examples of the alkynyl group include C₂₋₆ alkynyl such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like, inter alia ethynyl, 1-propynyl, 2-propynyl and the like are particularly preferred.

The alicyclic hydrocarbon group of the hydrocarbon group includes a saturated or unsaturated alicyclic hydrocarbon group such as a cycloalkyl group, a cycloalkenyl group, a cycloalkadienyl group and the like. As the cycloalkyl group, a C₃₋₉ cycloalkyl group is preferred, and examples thereof include cyclopropyl, cyclobutyl, cyclopentyl, cyclohexyl, cycloheptyl, cyclooctyl, cyclononyl and the like. Among these, a C₃₋₆ cycloalkyl group such as cyclopropyl, cyclobutyl, cyclopentyl and cyclohexyl is preferred. Examples of the cycloalkenyl group include a C₅₋₆ cycloalkenyl group such as 2-cyclopenten-1-yl, 3-cyclopenten-1-yl, 2-cyclohexen-1-yl, 3-cyclohexene-1-yl, 1-cyclobuten-1-yl and 1-cyclopenten-1-yl. Examples of the cycloalkadienyl group include a C₅₋₆ cycloalkadienyl group such as 2,4-cyclopentadien-1-yl, 2,4-cyclohexadien-1-yl and 2,5-cyclohexadien-1-yl.

The aryl group of the hydrocarbon group includes a C₆₋₁₆ monocyclic or fused polycyclic aromatic hydrocarbon group such as phenyl, naphthyl, anthryl, phenanthryl and acenaphthylenyl, inter alia, a C₆₋₁₀ aryl group such as phenyl, 1-naphthyl and 2-naphthyl is particularly preferred.

The substituent of the “optionally substituted hydrocarbon group” represented by R₁ includes an optionally substituted aryl group, an optionally substituted cycloalkyl group, an optionally substituted cycloalkenyl group, an optionally substituted heterocyclic group, an optionally substituted amino group, an optionally substituted hydroxy group, an optionally substituted thiol group, a halogen atom (e.g., fluorine, chlorine, bromine, iodine) and oxo etc., and the hydrocarbon group is optionally substituted with arbitrary 1 to 5 (preferably 1 to 3) of these substituents at a substitutable position. Examples of the aryl group of the optionally substituted aryl group include a C₆₋₁₆ aryl group such as phenyl, naphthyl, anthryl, phenanthryl and acenaphthylenyl, inter alia, a C₆₋₁₀ aryl group such as phenyl, 1-naphthyl and 2-naphthyl is preferred. The substituent of the optionally substituted aryl group includes a C₁₋₃ alkoxy group (e.g., methoxy, ethoxy, propoxy, etc.), a halogen atom (e.g., fluorine, chlorine, bromine, iodine), a C₁₋₃ alkyl group (e.g., methyl, ethyl, propyl, etc.) and the like, and the aryl group is optionally substituted with arbitrary 1 to 2 of these substituents. Examples of the cycloalkyl group of the optionally substituted cycloalkyl group include a C₃₋₇ cycloalkyl group such as cyclopropyl, cyclobutyl, cyclopentyl, cyclohexyl and cycloheptyl. As for the substituent of the optionally substituted cycloalkyl group and the number of the substituents, the same kind and number as in the substituent for the aforementioned optionally substituted aryl group may be exemplified. Examples of the cycloalkenyl group of the optionally substituted cycloalkenyl group include a C₃₋₆ cycloalkenyl group such as cyclopropenyl, cyclobutenyl, cyclopentenyl and cyclohexenyl. As for the substituent of the optionally substituted cycloalkenyl group and the number of the substituents, the same kind and number as in the substituent for the aforementioned optionally substituted aryl group may be exemplified. A heterocyclic group of the optionally substituted heterocyclic group includes an aromatic heterocyclic group and a saturated or unsaturated non-aromatic heterocyclic group (aliphatic heterocyclic group) containing at least one and preferably 1 to 4 hetero atoms selected from oxygen, sulfur and nitrogen as a ring system constituent atom (ring atom), and an aromatic heterocyclic group is preferred. Examples of the aromatic heterocyclic group include a 5- to 6-membered aromatic monocyclic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl, etc.) and an aromatic fused heterocyclic group in which 2 to 3 of 5- to 6-membered rings are fused (e.g., benzofuranyl, isobenzofuranyl, benzo[b]thienyl, indolyl, isoindolyl, 1H-indazolyl, benzimidazolyl, benzoxazolyl, 1,2-benzoisoxazolyl, benzothiazolyl, 1,2-benzoisothiazolyl, 1H-benzotriazolyl, quinolyl, isoquinolyl, cinnolyl, quinazolinyl, quinoxalinyl, phthalazinyl, naphthylizinyl, purinyl, pteridinyl, carbazolyl, α-carbolinyl, β-carbolinyl, γ-carbolinyl, acridinyl, phenoxazinyl, phenothiazinyl, phenazinyl, phenoxathinyl, thianthrenyl, phenanthridinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.), inter alia, a 5- to 6-membered aromatic monocyclic heterocyclic group such as furyl, thienyl, indolyl, isoindolyl, pyrazinyl, pyridyl and pyrimidinyl is preferred. Examples of the non-aromatic heterocyclic group include a 4- to 8-membered non-aromatic heterocyclic group such as oxiranyl, azetidinyl, oxetanyl, thietanyl, pyrrolidinyl, tetrahydrofuryl, thiolanyl, piperidyl, tetrahydropyranyl, morpholinyl, thiomorpholinyl and piperazinyl. The optionally substituted heterocyclic group may have 1 to 4, preferably 1 to 2 substituents, and such substituents include C₁₋₃ alkyl group (e.g., methyl, ethyl, propyl, etc.) and the like. As the substituent in the optionally substituted amino group (including amino group, mono- or di-substituted amino group), the optionally substituted hydroxy group and the optionally substituted thiol group, a lower (C₁₋₃) alkyl (e.g., methyl, ethyl, propyl, etc.) and the like are exemplified. Further, when the hydrocarbon group in the optionally substituted hydrocarbon group represented by R₁ is an alicyclic hydrocarbon group or an aryl group, the substituent may be also a C₁₋₃ alkyl group (e.g., methyl, ethyl, propyl, etc.).

In addition, as described above, R₁ may have an oxo group as a substituent, and a carboxylic acid acyl group which is such a hydrocarbon group substituted with oxo is included in R₁. Examples thereof include an optionally substituted C₁₋₆ acyl group (e.g., formyl, acetyl, propionyl, butyryl, isobutyryl, valeryl, isovaleryl, pivaloyl, hexanoyl, dimethylacetyl, trimethylacetyl, etc.) and the like. Further, the acyl group may have 1 to 5 substituents at a substitutable position, and the substituent includes a halogen atom (e.g., fluorine, chlorine, bromine).

In the formulae (I) and (Ia), the “optionally substituted hydrocarbon group” represented by R₂ and R₃ may include the group descried as the “optionally substituted hydrocarbon group” represented by R₁. However, an alkyl group, an aryl group and substituents thereof may be the group as follows. That is, as for the alkyl group of the “optionally substituted alkyl group”, a C₁₋₆ lower alkyl group (e.g., methyl, ethyl, n-propyl, isopropyl, butyl, isobutyl, sec-butyl, tert-butyl, pentyl, isopentyl, neopentyl, hexyl, isohexyl, etc.) is exemplified, and preferably a C₁₋₄ alkyl group such as methyl, ethyl, propyl, isopropyl, butyl, tert-butyl is exemplified. For example, these optionally substituted alkyl group may have 1 to 4 substituents, and such substituents include a halogen atom (e.g., fluorine, chlorine, bromine, iodine), C₁₋₄ lower alkoxy group (e.g., methoxy, ethoxy, propoxy, isopropoxy, butoxy, tert-butoxy, etc.) and the like.

The “optionally substituted aryl group” includes monocyclic or fused polycyclic aromatic hydrocarbon group such as phenyl, naphthyl, anthryl, phenanthryl and acenaphthylenyl, and among them, phenyl is particularly preferred. The substituent of the “optionally substituted aryl group” includes a halogen atom (e.g., fluorine, chlorine, bromine, iodine etc.), optionally substituted lower alkyl group, optionally substituted lower alkoxy group, an optionally substituted hydroxy group, nitro and cyano, and may be substituted with the same or different 1 to 3 (preferably 1 to 2) of these substituents. Examples of the lower alkyl include a C₁₋₄ alkyl group such as methyl, ethyl, n-propyl, isopropyl, n-butyl, isobutyl, sec-butyl and tert-butyl, inter alia, methyl and ethyl is particularly preferred. Examples of the lower alkoxy include a C₁₋₄ alkoxy group such as methoxy, ethoxy, n-propoxy, isopropoxy, n-butoxy, isobutoxy, sec-butoxy, tert-butoxy, inter alia, methoxy and ethoxy is particularly preferred. The substituent of the optionally substituted lower alkyl and the optionally substituted lower alkoxy includes a halogen atom (e.g., fluorine, chlorine, bromine, iodine etc.), and may be substituted with 1 to 5 at an arbitrary substitutable position. Examples of the substituent in the optionally substituted hydroxy group include a lower (C₁₋₄) alkyl group (e.g., methyl, ethyl, propyl, isopropyl, butyl, tert-butyl etc.), a C₃₋₆ cycloalkyl group (e.g., cyclopropyl, cyclobutyl, cyclopentyl, cyclohexyl, etc.), a C₆₋₁₀ aryl group (e.g., phenyl, 1-naphthyl, 2-naphthyl, etc.) and a C₇₋₁₂ aralkyl group (e.g., benzyl, phenethyl, etc.). Further, these substituents may be combined together with the adjacent substituent to form a ring, and when the aryl group of the “optionally substituted aryl group” represented by R₂ and R₃ is a phenyl group, a group represented by

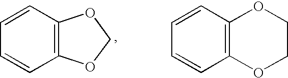

may be used, and furthermore, such groups may be substituted with 1 to 4 of lower (C₁₋₃) alkyl group (e.g., methyl, ethyl, propyl, isopropyl, etc.) and the like.

The heterocyclic group of the “optionally substituted heterocyclic group” represented by R₂ and R₃ includes the heterocyclic group described in detail for the “optionally substituted heterocyclic group” given as a substituent for the “optionally substituted hydrocarbon group” represented by R₁. Among those, 5- to 6-membered aromatic monocyclic heterocyclic ring such as furyl, thienyl, indolyl, isoindolyl, pyrazinyl, pyridyl, pyrimidyl and imidazolyl is particularly preferred. The substituent for the heterocyclic group includes C₁₋₃ alkyl (e.g., methyl, ethyl, propyl, etc.), and said heterocyclic ring may have 1 to 4 of such substituents.

Among the above, as for R₂ and R₃, an optionally substituted phenyl group is preferred, a substituted phenyl group is more preferred, and particularly, a phenyl group substituted with 1 to 3, preferably 1 to 2 of a halogen atom such as chlorine and bromine, lower (C₁₋₃) alkoxy or the like is preferred. Further, any one of R₂ and R₃ is preferably a hydrogen atom.

In the formula (I), the “group comprising an optionally esterified carboxyl group” represented by X′ includes an optionally esterified carboxyl group and a group having an optionally esterified carboxyl group. The optionally esterified carboxyl group includes the same group as that defined with respect to Y hereinafter.

The “group comprising an optionally substituted carbamoyl group” represented by X′ includes an optionally substituted carbamoyl group and a group having an optionally substituted carbamoyl group. The optionally substituted carbamoyl group includes the same group as that defined with respect to Y hereinafter.

The “group comprising an optionally substituted hydroxy group” represented by X′ includes an optionally substituted hydroxy group and a group having an optionally substituted hydroxy group. The optionally substituted hydroxy group includes the same group as that defined with respect to Y hereinafter.

The “group comprising an optionally substituted amino group” represented by X′ includes an optionally substituted amino group and a group having an optionally substituted amino group. The optionally substituted amino group includes the same group as that defined with respect to Y hereinafter.

The “group comprising an optionally substituted heterocyclic residue having a hydrogen atom which can be deprotonated” represented by X′ includes an optionally substituted heterocyclic residue having a hydrogen atom which can be deprotonated (i.e., having an active proton) and a group having an optionally substituted heterocyclic residue having a hydrogen atom which can be deprotonated. The optionally substituted heterocyclic residue having a hydrogen atom which can be deprotonated includes the same group as that defined with respect to Y hereinafter.

X′ includes a group represented by the formula (a):

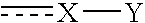

wherein, X is a bond, or divalent or trivalent atomic chain, Y is an optionally esterified carboxyl group, an optionally substituted carbamoyl group, an optionally substituted hydroxy group, an optionally substituted amino group, or an optionally substituted heterocyclic residue having a hydrogen atom which can be deprotonated, and the dotted line is a single or double bond.

In the formula (a), the “divalent atomic chain” represented by X may be any divalent chain having preferably 1 to 7, and more preferably 1 to 4 of atoms composing the linear portion, and may have a side chain.

Example thereof includes a group represented by

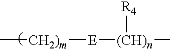

wherein, m and n represent an integer of 0, 1, 2 or 3, independently, E represents a bond or an oxygen atom, a sulfur atom, sulfoxide, sulfone, —N(R₅)—, —NHCO—, —CON(R₆)— or —NHCONH—. Herein, R₄ and R₆ represent a hydrogen atom, an optionally substituted lower alkyl group, an optionally substituted aralkyl group or an optionally substituted phenyl group. In addition, R₅ represents a hydrogen atom, a lower alkyl group, an aralkyl group or an acyl group.

The alkyl group of the “optionally substituted lower alkyl group” represented by R₄ and R₆ includes a C₁₋₆ linear or branched lower alkyl group (e.g., methyl, ethyl, n-propyl, isopropyl, n-butyl, isobutyl, tert-butyl, n-pentyl, isopentyl, neopentyl, etc.). The optionally substituted lower alkyl group may have 1 to 4, preferably 1 to 2 substituents, and examples of such substituents include an aromatic heterocyclic group (e.g., 5- to 6-membered aromatic heterocyclic ring containing 1 to 4 hetero atoms of N, O and S such as furyl, thienyl, indolyl, isoindolyl, pyrazinyl, pyridyl, pyrimidyl and imidazolyl), an optionally substituted amino group, an optionally substituted hydroxy group, an optionally substituted thiol group, an optionally esterified carboxyl group and a halogen atom (e.g., fluorine, chlorine, bromine, iodine). The substituent in the optionally substituted amino group (including amino group, mono- or di-substituted amino group), an optionally substituted hydroxy group and an optionally substituted thiol group includes lower (C₁₋₃) alkyl (e.g., methyl, ethyl, propyl, etc.). Examples of the optionally esterified carboxyl group include C₂₋₅ alkoxycarbonyl such as methoxycarbonyl ethoxycarbonyl, propoxycarbonyl, phenoxycarbonyl and 1-naphthoxycarbonyl, and C₇₋₁₁ aryloxycarbonyl, and preferably, methoxycarbonyl, ethoxycarbonyl and propoxycarbonyl are exemplified.

The aralkyl group of the “optionally substituted aralkyl group” represented by R₄ and R₆ includes a C₇-C₁₅ aralkyl group such as benzyl, naphthylmethyl, phenylpropyl and phenylbutyl. The optionally substituted aralkyl group may have 1 to 4, preferably 1 to 2 substituents, and such substituents include a halogen atom (e.g., fluorine, chlorine, bromine, iodine), a C₁₋₃ alkoxy group (e.g., methoxy, ethoxy, propoxy group), a hydroxy group, an amino group, a carboxyl group, a sulfhydryl group etc.

The substituent of the “optionally substituted phenyl group” represented by R₄ and R₆ includes a halogen atom (e.g., fluorine, chlorine, bromine, iodine), a C₁₋₃ alkoxy (e.g., methoxy, ethoxy, propoxy, etc.), C₁₋₃ alkyl (e.g., methyl, ethyl, propyl).

Provided that, R₄ may be different in every methylene chain.

In addition, examples of the “lower alkyl group” and the “aralkyl group” represented by R₅ include a C₁₋₄ lower alkyl group (e.g., methyl, ethyl, propyl, butyl, tert-butyl, etc.), a C₇₋₁₅ aralkyl group (e.g., benzyl, phenethyl, phenylpropyl, phenylbutyl, naphthylmethyl, etc.), respectively.

Examples of the “acyl group” represented by R₅ include a lower (C₁₋₆) alkanoyl group (e.g., formyl, acetyl, propionyl, butyryl, isobutyryl, valeryl, isovaleryl, pivaloyl, hexanoyl, etc.), a lower (C₃₋₇) alkenoyl group (e.g., acryloyl, methacryloyl, crotonoyl, isocrotonoyl, etc.), a C₄₋₇ cycloalkanecarbonyl group (e.g., a cyclopropanecarbonyl group, a cyclobutanecarbonyl group, a cyclopentanecarbonyl group, a cyclohexanecarbonyl group, etc.), a lower (C₁₋₄) alkanesulfonyl group (e.g., mesyl, ethanesulfonyl, propanesulfonyl, etc.), a C₇₋₁₄ aroyl group (e.g., benzoyl, p-toluoyl, 1-naphthoyl, 2-naphthoyl, etc.), a C₆₋₁₀ aryl lower (C₂₋₄) alkanoyl group (e.g., phenylacetyl, phenylpropionyl, hydroatropoyl, phenylbutyryl, etc.), a C₆₋₁₀ aryl lower (C₃₋₅) alkenoyl group (e.g., cinnamoyl, atropoyl, etc.), a C₆₋₁₀ arenesulfonyl group (e.g., benzenesulfonyl, a p-toluenesulfonyl group, etc.).

Further, X may be a carbon chain having a double bond or -L-CH(OH)— (L represents a bond or a linear or branched alkylene chain). Examples of the “carbon chain having a double bond” include a carbon chain having, preferably 1 to 7, more preferably 1 to 4 of carbon atoms constituting the linear portion, and may also have a side chain. The double bond in the carbon chain is contained in any one or both of a linear portion and a branched portion, and preferably contained in the linear portion. Further, the number of double bonds contained in the carbon chain is not particularly limited, if possible, but 1 to 2 is preferred.

Examples of the carbon chain having double bond include methine, vinylene, propenylene, butenylene, butadienylene, methylpropenylene, ethylpropenylene, propylpropenylene, methylbutenylene, ethylbutenylene, propylbutenylene, methylbutadienylene, ethylbutadienylene, propylbutadienylene, pentenylene, hexenylene, heptenylene, pentadienylene, hexadienylene, heptadienylene and the like, and preferably, methine, vinylene, propenylene, butenylene and butadienylene are exemplified. Herein, when the carbon chain is trivalent, the carbon chain forms a double bond with a substitutable carbon atom on the ring of ring J′.

Examples of the “linear or branched alkylene chain” represented by L include a linear or branched C₁₋₆ alkylene chain, for example, a divalent group such as methylene, ethylene, trimethylene, tetramethylene, pentamethylene, hexamethylene, heptamethylene, propylene, ethylmethylene, ethylethylene, propylethylene, butylethylene, methyltetramethylene and methyltrimethylene, and preferably, a C₁₋₃ chain such as methylene, ethylene, trimethylene and propylene are exemplified.

Among these, X′ is preferably a group represented by the formula (b):

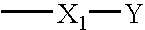

wherein X₁ represents a bond or divalent atomic chain, Y represents an optionally esterified carboxyl group, an optionally substituted carbamoyl group, an optionally substituted hydroxy group, an optionally substituted amino group or an optionally substituted heterocyclic group having a hydrogen atom which can be deprotonated.

In the formula (b), as for the divalent atomic chain represented by X₁, the same as in the divalent atomic chain defined with respect to the aforementioned X may be exemplified.

In the formulae (a) and (b), the “divalent atomic chain” represented by X or X₁ includes a linear or branched alkylene chain having preferably 1 to 7 (more preferably 1 to 4) of carbon atoms constituting the linear portion. Examples of the alkylene chain include a divalent group such as methylene, ethylene, trimethylene, tetramethylene, pentamethylene, hexamethylene, heptamethylene, propylene, ethylmethylene, ethylethylene, propylethylene, butylethylene, methyltetramethylene and methyltrimethylene, and preferably, a C₁₋₄ chain such as methylene, ethylene, trimethylene and propylene is exemplified.

In the formulae (a) and (b), the “optionally esterified carboxyl group” represented by Y includes a C₂₋₇ lower alkoxycarbonyl (e.g., methoxycarbonyl, ethoxycarbonyl, propoxycarbonyl, isopropoxycarbonyl, butoxycarbonyl, tert-butoxycarbonyl, sec-butoxycarbonyl, pentyloxycarbonyl, isopentyloxycarbonyl, neopentyloxycarbonyl, etc.), C₇₋₁₄ aryloxycarbonyl (e.g., phenoxycarbonyl, 1-naphthoxycarbonyl) and C₈₋₁₂ aralkyloxycarbonyl (e.g., benzyloxycarbonyl, etc.). Among these, a carboxyl group, methoxycarbonyl, and ethoxycarbonyl are preferred.

The substituent of the “optionally substituted carbamoyl group” represented by Y includes an optionally substituted lower (C₁₋₆) alkyl (e.g., methyl, ethyl, n-propyl, isopropyl, butyl, isobutyl, sec-butyl, tert-butyl, pentyl, isopentyl, neopentyl, hexyl, isohexyl, etc.), an optionally substituted C₃₋₆ cycloalkyl (e.g., cyclopropyl, cyclobutyl, cyclopentyl, cyclohexyl, etc.), an optionally substituted C₆₋₁₄ aryl group (e.g., phenyl, 1-naphthyl, 2-naphthyl, etc.) and an optionally substituted C₇₋₁₁ aralkyl group (e.g., benzyl, phenethyl, etc.), and the carbamoyl group may be substituted with the same or different 1 to 2 of these substituents. The substituent in the optionally substituted lower (C₁₋₆) alkyl and optionally substituted C₃₋₆ cycloalkyl includes a carboxyl group optionally esterified with lower (C₁₋₅) alkyl (e.g., methyl, ethyl, propyl, isopropyl, butyl, tert-butyl, pentyl, isopentyl, neopentyl), a 5- to 6-membered aromatic heterocyclic group containing 1 to 4 hetero atoms (e.g., furyl, thienyl, indolyl, isoindolyl, pyrazinyl, pyridyl, pyrimidyl, imidazolyl, etc.), an amino group, a hydroxy group and a phenyl group, and the same or different 1 to 3 of these substituents may substitute. The substituent of the optionally substituted aryl group and the optionally substituted aralkyl group includes a halogen atom (e.g., fluorine, chlorine, bromine, iodine), and carboxyl group optionally esterified with a lower (C₁₋₄) alkyl group (e.g., methyl, ethyl, propyl, isopropyl, butyl, tert-butyl, etc.). In addition, in the optionally substituted carbamoyl group, the two substituents on the nitrogen atoms may be combined together with the nitrogen atoms to form a cyclic amino group, and examples of such cyclic amino group include 1-azetidinyl, 1-pyrrolidinyl, piperidino, morpholino, 1-piperazinyl and the like. Further, the cyclic amino group may also have a substituent.

The substituent of the “optionally substituted hydroxy group” represented by Y includes, for example, lower (C₁₋₄) alkyl (e.g., methyl, ethyl, propyl, isopropyl, butyl, tert-butyl, etc.), a C₃₋₆ cycloalkyl group (e.g., cyclopropyl, cyclobutyl, cyclopentyl, cyclohexyl, etc.), an optionally substituted C₆₋₁₀ aryl group (e.g., phenyl, 1-naphthyl, 2-naphthyl, etc.) and an optionally substituted C₇₋₁₁ aralkyl group (e.g., benzyl, phenethyl, etc.). The substituent of the optionally substituted aryl group and the optionally substituted aralkyl group includes a halogen atom (e.g., fluorine, chlorine, bromine, iodine), carboxyl group optionally esterified with a lower (C₁₋₄) alkyl group (e.g., methyl, ethyl, propyl, isopropyl, butyl, tert-butyl, etc.), and the like.

The “optionally substituted amino group” represented by Y includes a mono-substituted and di-substituted amino group, and examples of such substituent include lower (C₁₋₄) alkyl (e.g., methyl, ethyl, propyl, isopropyl, butyl, tert-butyl, etc.), a C₃₋₆ cycloalkyl group (e.g., cyclopropyl, cyclobutyl, cyclopentyl, cyclohexyl, etc.), an optionally substituted C₆₋₁₀ aryl group (e.g., phenyl, 1-naphthyl, 2-naphthyl, etc.), an optionally substituted C₇₋₁₁ aralkyl group (e.g., benzyl, phenethyl, etc.) and the like. The substituent of the optionally substituted aryl group and the optionally substituted aralkyl group includes a halogen atom (e.g., fluorine, chlorine, bromine, iodine), carboxyl group optionally esterified with a lower (C₁₋₄) alkyl group (e.g., methyl, ethyl, propyl, isopropyl, butyl, tert-butyl, etc.) and the like, and 1 to 4, preferably 1 to 2 of these substituents may be possessed. In addition, two of the substituents on the nitrogen atom may be combined together with the nitrogen atom to form a cyclic amino group, and examples of such cyclic amino group include 1-azetidinyl, 1-pyrrolidinyl, piperidino, morpholino, 1-piperazinyl. In addition, the cyclic amino group may also have a further substituent.

The heterocyclic residue of the “optionally substituted heterocyclic group having a hydrogen atom which can be deprotonated” represented by Y includes a 5- to 7-membered (preferably 5-membered) monocyclic heterocyclic residue having at least one selected from N, S and O (preferably a nitrogen-containing heterocyclic residue), which has a hydrogen atom that can eliminate to form a proton. Examples thereof include tetrazol-5-yl or a group represented by the formula:

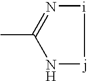

wherein, i represents —O— or —S—, j represents >C═O, >C═S or >S(O)₂, (among these, 2,5-dihydro-5-oxo-1,2,4-oxadiazol-3-yl, 2,5-dihydro-5-thioxo-1,2,4-oxadiazol-3-yl and 2,5-dihydro-5-oxo-1,2,4-thiadiazol-3-yl are preferred).

The above heterocyclic residue may be protected with an optionally substituted lower alkyl group (preferably C₁₋₄ alkyl) or an acyl group. Examples of the optionally substituted-lower alkyl group include C₁₋₄ alkyl optionally substituted with 1) phenyl optionally substituted with C₁₋₃ alkyl, nitro or C₁₋₃ alkoxy or 2) C₁₋₃ alkoxy (e.g., methyl, triphenylmethyl, methoxymethyl, ethoxymethyl, p-methoxybenzyl, p-nitrobenzyl, etc.). Examples of the acyl group include lower (C₂₋₅) alkanoyl, benzoyl and the like.

Among these, X′ is preferably an alkyl group substituted with an optionally esterified carboxyl group, an alkyl group substituted with an optionally substituted heterocyclic residue having a hydrogen which can be deprotonated or an alkyl group substituted with an optionally substituted carbamoyl group.

In the formula (I), the heterocyclic ring represented by Ring A includes a heterocyclic group described in detail with respect to the substituent of the hydrocarbon group represented by R₁. Among them, a group represented below is preferred.

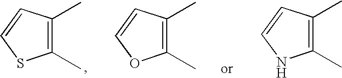

The substituent of the “optionally substituted benzene ring” and “optionally substituted heterocyclic ring” represented by Ring A includes a halogen atom (e.g., fluorine, chlorine, bromine, iodine), an optionally substituted C₁₋₄ lower alkyl group (e.g., methyl, ethyl, propyl, butyl, tert-butyl, etc.), an optionally substituted C₁₋₄ lower alkoxy group (e.g., methoxy, ethoxy, propoxy, isopropoxy, butoxy, tert-butoxy, etc.), a hydroxy group, a nitro group and cyano. Ring A may have 1 to 3, preferably 1 to 2 of these substituents. Further, these substituents may be combined together with the adjacent substituents to form a ring. The substituent of the optionally substituted lower alkyl group and the optionally substituted lower alkoxy group includes a halogen atom (e.g., fluorine, chlorine, bromine, iodine) and the like, and 1 to 3 of substituents may be present at an arbitrary position. Ring A is preferably substituted with methoxy or a chlorine atom, and Ring A substituted with a chlorine atom is particularly preferred.

In the formula (Ia), the substituent of the “optionally substituted benzene ring” represented by Ring B includes a halogen atom (e.g., fluorine, chlorine, bromine, iodine), an optionally substituted C₁₋₄ lower alkyl group (e.g., methyl, ethyl, propyl, butyl, tert-butyl, etc.), an optionally substituted C₁₋₄ lower alkoxy group (e.g., methoxy, ethoxy, propoxy, isopropoxy, butoxy, tert-butoxy, etc.), a hydroxy group, a nitro group and cyano. Ring B may have 1 to 3, preferably 0.1 to 2 of these substituents. Further, these substituents may be combined together with the adjacent substituents to form a ring. The substituent of the optionally substituted lower alkyl group and the optionally substituted lower alkoxy group includes a halogen atom (e.g., fluorine, chlorine, bromine, iodine) and the like, and 1 to 3 of substituents may be present at an arbitrary position. Ring B is preferably substituted with methoxy or a chlorine atom, and Ring B substituted with a chlorine atom is particularly preferred.

In the formula (I), the heterocyclic ring in the “7- to 8-membered heterocyclic ring containing 3 or less hetero atoms as ring constituent atoms” represented by the ring J′ includes, for example, a saturated or unsaturated 7- or 8-membered heterocyclic ring containing at least one selected from O, S(O)_{q} (q represents 0, 1 or 2) and N. However, the hetero atoms in the atoms constituting the ring of said heterocyclic ring (ring constituent atom) are three or less.

Further, Ring J′ may have 1 to 2 substituents at a substitutable position in addition to a group represented by R₁, R₂, R₃ and X′. When the substituent is attached to a nitrogen atom on Ring J′, examples of the substituent include an alkyl group (e.g., C₁₋₆ alkyl such as methyl, ethyl, n-propyl, isopropyl, n-butyl, isobutyl, tert-butyl, n-pentyl, isopentyl, neopentyl, etc.), an acyl group (e.g., C₁₋₄ acyl group such as formyl, acetyl, propionyl, butyroyl, etc.). The alkyl group or acyl group may further be substituted with 1 to 5 of halogen atoms (e.g., fluorine, chlorine, bromine, iodine). Further, when the substituent is attached to a carbon atom on the Ring J′, examples of the substituent include oxo, thioxo, an optionally substituted hydroxy group and an optionally substituted amino group. As for the optionally substituted hydroxy group and the optionally substituted amino group, the same as in the “optionally substituted hydroxy group” and the “optionally substituted amino group” defined as Y above may be exemplified.

Ring J′ is preferably substituted with oxo or thioxo at a substitutable position in addition to the group represented by R₁, R₂, R₃ and X′.

Examples of a fused ring comprising Ring A and ring J′ include

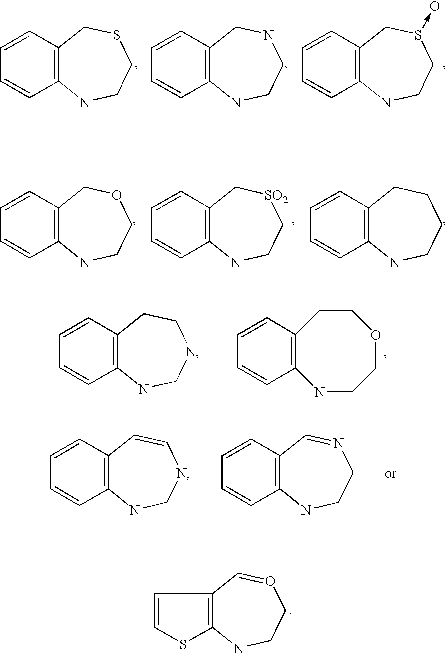

The formula (I) is preferably a group represented by the formula (I′)

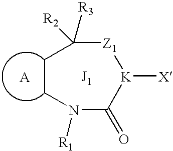

wherein, R₁, R₂, R₃, X′ and Ring A are as defined above, and Ring J₁ represents a 7-membered heterocyclic ring, Z₁ represents —N(R₇)— (R₇ represents a hydrogen atom, an alkyl group or an acyl group), —S(O)_{q} (q represents 0, 1 or 2), —CH₂— or —O—, K represents C or N, and G represents O or S.

In the formula (I′) above, the alkyl group represented by R₇ includes a C₁₋₆ linear or branched lower alkyl group (e.g., methyl, ethyl, n-propyl, isopropyl, n-butyl, isobutyl, tert-butyl, n-pentyl, isopentyl, neopentyl, etc.), which may be substituted with 1 to 5 of halogen atoms (e.g., fluorine, chlorine, bromine, iodine).

Examples of the acyl group represented by R₇ include a C₁₋₄ acyl group (e.g., formyl, acetyl, propionyl, butyroyl, etc.), which may be substituted with 1 to 5 of halogen atoms (e.g., fluorine, chlorine, bromine, iodine).

In the formula (I′), Z₁ is preferably S(O)_{q} (q represents 0, 1 or 2) or O. Further, K is preferably C and G is preferably O.

As for the formula (I′), a compound represented by the formula (I″)

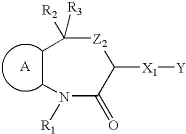

wherein, R₁, R₂, R₃, X₁, Y and Ring A are as defined above, and Z₂ represents S(O)_{q} (q represent 0, 1 or 2) or O, is more preferred.

The compound represented by the formula (I) is preferably the compound represented by the formula (Ia)

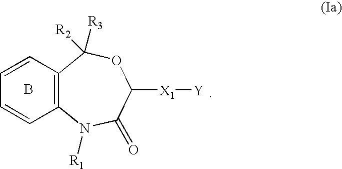

The compound of formula (Ia) may be also a compound represented by the formula (Ia′)

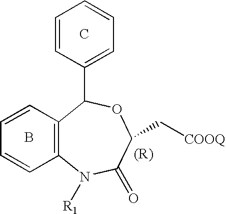

wherein, R₁ and Ring B are as defined above, and Q represents a hydrogen atom or a metal ion, Ring C represents an optionally substituted phenyl group. In the formula, the substituents at 3- and 5-position represent trans which faces the opposite direction relative to the plane of the 7-membered ring, and (R) represents R-configuration.

In the formula (Ia′), the metal ion represented by Q includes a sodium ion, a potassium ion, a calcium ion, an aluminum ion and the like, inter alia, a sodium ion and a potassium ion are preferred.

The substituent of the “optionally substituted phenyl group” represented by Ring C includes the same group as the substituent of the “optionally substituted aryl group” described as an example of the “optionally substituted hydrocarbon group” defined with respect to R₂ and R₃ above.

Examples of the salt of the compound represented by the formula (I) include pharmacologically acceptable salts such as an inorganic salt such as hydrochloride, hydrobromide, sulfate, nitrate and phosphate, an organic acid salt such as acetate, tartrate, citrate, fumarate, maleate, toluenesulfonate and methanesulfonate, a metal salt such as sodium salt, potassium salt, calcium salt and aluminum salt, and a salt with base such as triethylamine salt, guanidine salt, ammonium salt, hydrazine salt, quinine salt and cinchonine salt. Among these, a sodium salt is preferred.

Specific examples of the compound represented by the formula (I) includes below:
(3R,5S)-7-cyano-5-(2,3-dimethoxyphenyl)-1-neopentyl-2-oxo-1,2,3,5-tetrahydro-4,1-benzoxazepine-3-acetic acid,
(3R,5S)-7-cyano-5-(2,4-dimethoxyphenyl)-1-neopentyl-2-oxo-1,2,3,5-tetrahydro-4,1-benzoxazepine-3-acetic acid,
(3R,5S)-7-cyano-5-(2,3-methylenedioxyphenyl)-1-neopentyl-2-oxo-1,2,3,5-tetrahydro-4,1-benzoxazepine-3-acetic acid,
(3R,5S)-7-cyano-5-(2,3-ethylenedioxyphenyl)-1-neopentyl-2-oxo-1,2,3,5-tetrahydro-4,1-benzoxazepine-3-acetic acid,
(3R,5S)-7-cyano-5-(2,3-dimethoxyphenyl)-1-isobutyl-2-oxo-1,2,3,5-tetrahydro-4,1-benzoxazepine-3-acetic acid,
(3R,5S)-7-cyano-5-(2,4-dimethoxyphenyl)-1-isobutyl-2-oxo-1,2,3,5-tetrahydro-4,1-benzoxazepine-3-acetic acid,
(3R,5S)-7-cyano-5-(2,3-methylenedioxyphenyl)-1-isobutyl-2-oxo-1,2,3,5-tetrahydro-4,1-benzoxazepine-3-acetic acid,
(3R,5S)-7-cyano-5-(2,3-ethylenedioxyphenyl)-1-isobutyl-2-oxo-1,2,3,5-tetrahydro-4,1-benzoxazepine-3-acetic acid,
(3R,5S)-7-chloro-5-(2,3-dimethoxyphenyl)-1-neopentyl-2-oxo-1,2,3,5-tetrahydro-4,1-benzoxazepine-3-acetic acid,
(3R,5S)-7-chloro-5-(2,4-dimethoxyphenyl)-1-neopentyl-2-oxo-1,2,3,5-tetrahydro-4,1-benzoxazepine-3-acetic acid,
(3R,5S)-7-chloro-5-(2,3-methylenedioxyphenyl)-1-neopentyl-2-oxo-1,2,3,5-tetrahydro-4,1-benzoxazepine-3-acetic acid,
(3R,5S)-7-chloro-5-(2,3-ethylenedioxyphenyl)-1-neopentyl-2-oxo-1,2,3,5-tetrahydro-4,1-benzoxazepine-3-acetic acid,
(3R,5S)-7-chloro-5-(2,3-dimethoxyphenyl)-1-isobutyl-2-oxo-1,2,3,5-tetrahydro-4,1-benzoxazepine-3-acetic acid,
(3R,5S)-7-chloro-5-(2,4-dimethoxyphenyl)-1-isobutyl-2-oxo-1,2,3,5-tetrahydro-4,1-benzoxazepine-3-acetic acid,
(3R,5S)-7-chloro-5-(2,3-methylenedioxyphenyl)-1-isobutyl-2-oxo-1,2,3,5-tetrahydro-4,1-benzoxazepine-3-acetic acid,
(3R,5S)-7-chloro-5-(2,3-ethylenedioxyphenyl)-1-isobutyl-2-oxo-1,2,3,5-tetrahydro-4,1-benzoxazepine-3-acetic acid,
(3R,5S)-7-cyano-5-(2,3-dimethoxyphenyl)-1-neopentyl-2-oxo-1,2,3,5-tetrahydro-4,1-benzothiazepine-3-acetic acid,
(3R,5S)-7-cyano-5-(2,4-dimethoxyphenyl)-1-neopentyl-2-oxo-1,2,3,5-tetrahydro-4,1-benzothiazepine-3-acetic acid,
(3R,5S)-7-cyano-5-(2,3-methylenedioxyphenyl)-1-neopentyl-2-oxo-1,2,3,5-tetrahydro-4,1-benzothiazepine-3-acetic acid,
(3R,5S)-7-cyano-5-(2,3-ethylenedioxyphenyl)-1-neopentyl-2-oxo-1,2,3,5-tetrahydro-4,1-benzothiazepine-3-acetic acid,
(3R,5S)-7-cyano-5-(2,3-dimethoxyphenyl)-1-isobutyl-2-oxo-1,2,3,5-tetrahydro-4,1-benzothiazepine-3-acetic acid,
(3R,5S)-7-cyano-5-(2,4-dimethoxyphenyl)-1-isobutyl-2-oxo-1,2,3,5-tetrahydro-4,1-benzothiazepine-3-acetic acid,
(3R,5S)-7-cyano-5-(2,3-methylenedioxyphenyl)-1-isobutyl-2-oxo-1,2,3,5-tetrahydro-4,1-benzothiazepine-3-acetic acid,
(3R,5S)-7-cyano-5-(2,3-ethylenedioxyphenyl)-1-isobutyl-2-oxo-1,2,3,5-tetrahydro-4,1-benzothiazepine-3-acetic acid,
(3R,5S)-7-chloro-5-(2,3-dimethoxyphenyl)-1-neopentyl-2-oxo-1,2,3,5-tetrahydro-4,1-benzothiazepine-3-acetic acid,
(3R,5S)-7-chloro-5-(2,4-dimethoxyphenyl)-1-neopentyl-2-oxo-1,2,3,5-tetrahydro-4,1-benzothiazepine-3-acetic acid,
(3R,5S)-7-chloro-5-(2,3-methylenedioxyphenyl)-1-neopentyl-2-oxo-1,2,3,5-tetrahydro-4,1-benzothiazepine-3-acetic acid,
(3R,5S)-7-chloro-5-(2,3-ethylenedioxyphenyl)-1-neopentyl-2-oxo-1,2,3,5-tetrahydro-4,1-benzothiazepine-3-acetic acid,
(3R,5S)-7-chloro-5-(2,3-dimethoxyphenyl)-1-isobutyl-2-oxo-1,2,3,5-tetrahydro-4,1-benzothiazepine-3-acetic acid,
(3R,5S)-7-chloro-5-(2,4-dimethoxyphenyl)-1-isobutyl-2-oxo-1,2,3,5-tetrahydro-4,1-benzothiazepine-3-acetic acid,
(3R,5S)-7-chloro-5-(2,3-methylenedioxyphenyl)-1-isobutyl-2-oxo-1,2,3,5-tetrahydro-4,1-benzothiazepine-3-acetic acid,
(3R,5S)-7-chloro-5-(2,3-ethylenedioxyphenyl)-1-isobutyl-2-oxo-1,2,3,5-tetrahydro-4,1-benzothiazepine-3-acetic acid,
(3R,5S)-7-cyano-5-(2-chlorophenyl)-1-neopentyl-2-oxo-1,2,3,5-tetrahydro-4,1-benzoxazepine-3-acetic acid,
(3R,5S)-7-cyano-5-(2-chlorophenyl)-1-isobutyl-2-oxo-1,2,3,5-tetrahydro-4,1-benzoxazepine-3-acetic acid,
(3R,5S)-7-chloro-5-(2-chlorophenyl)-1-neopentyl-2-oxo-1,2,3,5-tetrahydro-4,1-benzoxazepine-3-acetic acid,
(3R,5S)-7-chloro-5-(2-chlorophenyl)-1-isobutyl-2-oxo-1,2,3,5-tetrahydro-4,1-benzoxazepine-3-acetic acid,
(3R,5S)-7-cyano-5-(2-chlorophenyl)-1-neopentyl-2-oxo-1,2,3,5-tetrahydro-4,1-benzothiazepine-3-acetic acid,
(3R,5S)-7-cyano-5-(2-chlorophenyl)-1-isobutyl-2-oxo-1,2,3,5-tetrahydro-4,1-benzothiazepine-3-acetic acid,
(3R,5S)-7-chloro-5-(2-chlorophenyl)-1-neopentyl-2-oxo-1,2,3,5-tetrahydro-4,1-benzothiazepine-3-acetic acid,
(3R,5S)-7-chloro-5-(2-chlorophenyl)-1-isobutyl-2-oxo-1,2,3,5-tetrahydro-4,1-benzothiazepine-3-acetic acid,
(3R,5S)-7-chloro-5-(4-ethoxy-2-methoxyphenyl)-1-neopentyl-2-oxo-1,2,3,5-tetrahydro-4,1-benzoxazepine-3-acetic acid,
(3R)-7-chloro-5-(2-chlorophenyl)-2,3-dihydro-1-isobutyl-2-oxo-1H-1,4-benzodiazepine-3-acetic acid,
(3R,5S)-7-chloro-5-(2-chlorophenyl)-2,3,4,5-tetrahydro-1-isobutyl-2-oxo-1H-1,4-benzodiazepine-3-acetic acid,
N-[[(3R,5S)-7-chloro-5-(2-chlorophenyl)-1-neopentyl-2-oxo-1,2,3,5-tetrahydro-4,1-benzothiazepine-3-yl]-acetyl]glycine,
(3R,5S)-7-chloro-5-(2-chlorophenyl)-3-dimethylaminocarbonylmethyl-1-neopentyl-2-oxo-1,2,3,5-tetrahydro-4,1-benzothiazepine,
7-chloro-5-(2-chlorophenyl)-1-isobutyl-2-oxo-2,3,4,5-tetrahydro-1H-[1]-benzazepine-3-acetic acid,
(3R,5S)-7-chloro-5-(2-chlorophenyl)-1-neopentyl-1,2,3,5-tetrahydro-2-thioxo-4,1-benzoxazepine-3-acetic acid,
(3R,5S)-7-chloro-5-(2-chlorophenyl)-1-neopentyl-2-oxo-1,2,3,5-tetrahydro-4,1-thieno[2,3-e]oxazepine-3-acetic acid,
(3R,5S)-7-chloro-5-(2-methoxyphenyl)-1-neopentyl-2-oxo-1,2,3,5-tetrahydro-4,1-thieno[2,3-e]oxazepine-3-acetic acid,
(3R,5S)-7-chloro-1-isobutyl-5-(2-methoxyphenyl)-2-oxo-1,2,3,5-tetrahydro-4,1-thieno[2,3-e]oxazepine-3-acetic acid,
(3R,5S)-7-chloro-5-(3-hydroxy-2-methoxyphenyl)-1-neopentyl-2-oxo-1,2,3,5-tetrahydro-4,1-benzothiazepine-3-acetic acid,
(3R,5S)-7-chloro-5-(4-hydroxy-2-methoxyphenyl)-1-neopentyl-2-oxo-1,2,3,5-tetrahydro-4,1-benzothiazepine-3-acetic acid,
(3R,5S)-7-chloro-5-(3-hydroxy-2-methoxyphenyl)-1-isobutyl-2-oxo-1,2,3,5-tetrahydro-4,1-benzothiazepine-3-acetic acid,
(3R,5S)-7-chloro-5-(4-hydroxy-2-methoxyphenyl)-1-isobutyl-2-oxo-1,2,3,5-tetrahydro-4,1-benzothiazepine-3-acetic acid,
(3R,5S)-7-chloro-5-(3-ethoxy-2-methoxyphenyl)-1-neopentyl-2-oxo-1,2,3,5-tetrahydro-4,1-benzothiazepine-3-acetic acid,
(3R,5S)-7-chloro-5-(4-ethoxy-2-methoxyphenyl)-1-neopentyl-2-oxo-1,2,3,5-tetrahydro-4,1-benzothiazepine-3-acetic acid,
(3R,5S)-7-chloro-5-(3-ethoxy-2-methoxyphenyl)-1-isobutyl-2-oxo-1,2,3,5-tetrahydro-4,1-benzothiazepine-3-acetic acid,
(3R,5S)-7-chloro-5-(4-ethoxy-2-methoxyphenyl)-1-isobutyl-2-oxo-1,2,3,5-tetrahydro-4,1-benzothiazepine-3-acetic acid,
(3R,5S)-7-chloro-5-(2-chloro-3-methoxyphenyl)-1-neopentyl-2-oxo-1,2,3,5-tetrahydro-4,1-benzothiazepine-3-acetic acid,
(3R,5S)-7-chloro-5-(2-chloro-4-methoxyphenyl)-1-neopentyl-2-oxo-1,2,3,5-tetrahydro-4,1-benzothiazepine-3-acetic acid,
(3R,5S)-7-chloro-5-(2-chloro-3-methoxyphenyl)-1-isobutyl-2-oxo-1,2,3,5-tetrahydro-4,1-benzothiazepine-3-acetic acid,
(3R,5S)-7-chloro-5-(2-chloro-4-methoxyphenyl)-1-isobutyl-2-oxo-1,2,3,5-tetrahydro-4,1-benzothiazepine-3-acetic acid,
(3R,5S)-7-chloro-5-(2-chloro-3-hydroxyphenyl)-1-neopentyl-2-oxo-1,2,3,5-tetrahydro-4,1-benzothiazepine-3-acetic acid,
(3R,5S)-7-chloro-5-(2-chloro-4-hydroxyphenyl)-1-neopentyl-2-oxo-1,2,3,5-tetrahydro-4,1-benzothiazepine-3-acetic acid,
(3R,5S)-7-chloro-5-(2-chloro-3-hydroxyphenyl)-1-isobutyl-2-oxo-1,2,3,5-tetrahydro-4,1-benzothiazepin-3-acetic acid,
(3R,5S)-7-chloro-5-(2-chloro-4-hydroxyphenyl)-1-isobutyl-2-oxo-1,2,3,5-tetrahydro-4,1-benzothiazepine-3-acetic acid; and salts thereof.

The compounds represented by the formula (I) and the salts thereof [hereinafter, sometimes, abbreviated as Compound (I) including salts] are disclosed in, for example, EP-A-567026, WO95/21834 (Japanese Patent Application No. 6-15531), EP-A-645377 (Japanese Patent Application No. 6-229159), EP-A-645378 (Japanese Patent Application No. 6-229160), and can be prepared according to the disclosure of these publications.

The compound represented by the formula (I) is preferably the compound represented by the formula (Ib):

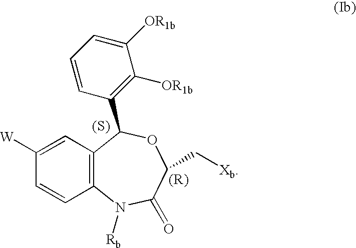

Preferable examples of the compound represented by the formula (Ib) include: the compound wherein R_{b} is a C₁₋₆ alkyl group which may have 1 to 3 substituents selected from hydroxy group, acetyloxy, propionyloxy, t-butoxycarbonyloxy, palmitoyloxy, dimethylaminoacetyloxy and 2-aminopropionyloxy; the compound wherein R_{b} is a branched C₃₋₆ alkyl group which may have 1 to 3 substituents selected from hydroxy group, acetyloxy, propionyloxy, t-butoxycarbonyloxy, palmitoyloxy, dimethylaminoacetyloxy and 2-aminopropionyloxy; the compound wherein R_{b} is 2,2-dimethyl-3-hydroxypropyl, 3-hydroxy-2-hydroxymethyl-2-methylpropyl, 3-acetoxy-2,2-dimethylpropyl, 3-acetoxy-2-hydroxymethyl-2-methylpropyl or 3-acetoxy-2-acetoxymethyl-2-methylpropyl;

the compound wherein R_{1b} is methyl;
the compound wherein W is chlorine atom;
the compound wherein X_{b} is a group represented by the formula:

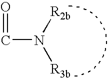

wherein, R_{2b} and R_{3b} are each a hydrogen atom, an optionally substituted hydrocarbon group, an optionally substituted heterocyclic group or an acyl group, or R_{2b} and R_{3b} may be combined together with the adjacent nitrogen atom to form an optionally substituted 5- or 6-membered nitrogen-containing heterocyclic ring optionally containing 1 to 3 hetero atoms selected from a nitrogen atom, a sulfur atom and an oxygen atom as ring constituent atoms;
the compound wherein as for a group represented by X_{b},

R_{2b} is a hydrogen atom or a C₁₋₇ alkyl group,

R_{3b} is (1) a hydrocarbon group selected from (a) C₁₋₇ alkyl, (b) C₃₋₇ cycloalkyl, (c) C₂₋₆ alkenyl, (d) C₆₋₁₀ aryl and (e) C₆₋₁₀ aryl-C₁₋₄ alkyl [wherein, (a) C₁₋₇ alkyl, (b) C₃₋₇ cycloalkyl and (c) C₂₋₆ alkenyl may be respectively substituted with 1 to 4 substituents selected from (i) carboxyl group optionally esterified with C₁₋₆ alkyl or C₆₋₁₀ aryl-C₁₋₄ alkyl, (ii) phosphate group optionally mono- or di-substituted with C₁₋₆ alkyl or C₂₋₇ alkanoyloxy-C₁₋₆ alkyl, (iii) a sulfonate group, (iv) sulfonamide group optionally substituted with C₁₋₆ alkyl or C₆₋₁₀ aryl-C₁₋₄ alkyl, (v) hydroxy group optionally alkylated with C₁₋₃ alkyl, (vi) sulfhydryl group optionally alkylated with C₁₋₃ alkyl, (vii) a carbamoyl group, (viii) phenyl group optionally substituted with 1 to 5 substituents selected from a hydroxy group, a chlorine atom, a fluorine atom, aminosulfonyl and amino group optionally mono- or di-substituted with C₁₋₃ alkyl, (ix) amino group optionally mono- or di-substituted with C₁₋₃ alkyl, (x) cyclic amino group derived from piperidine, pyrrolidine, morpholine, thiomorpholine, piperazine, 4-methylpiperazine, 4-benzylpiperazine, 4-phenylpiperazine, 1,2,3,4-tetrahydroisoquinoline or phthalimide, which may be substituted with C₁₋₃ alkyl, benzyl or phenyl and (xi) a 5- to 6-membered aromatic heterocyclic group derived from pyridine, imidazole, indole or tetrazole; and

(d) C₆₋₁₀ aryl and (e) C₆₋₁₀ aryl-C₁₋₄ alkyl may be respectively substituted with 1 to 4 substituents selected from (i) carboxyl group optionally esterified with C₁₋₄ alkyl, (ii) phosphate group optionally mono- or di-substituted with C₁₋₆ alkyl or C₂₋₇ alkanoyloxy-C₁₋₆ alkyl, (iii) a sulfonate group, (iv) a C₁₋₄ alkylsulfonyl, C₆₋₁₀ arylsulfonyl or C₆₋₁₀ aryl-C₁₋₄ alkylsulfonyl group, (v) sulfonamide group optionally substituted with C₁₋₆ alkyl or C₆₋₁₀ aryl-C₁₋₄ alkyl, (vi) C₁₋₃ alkyl group optionally substituted with carboxyl group optionally esterified with C₁₋₄ alkyl, phosphate group optionally mono- or di-substituted with C₁₋₆ alkyl, a sulfonate group, C₁₋₄ alkylsulfonyl, C₆₋₁₀ arylsulfonyl or C₆₋₁₀ aryl-C₁₋₄ alkylsulfonyl, sulfonamide group optionally substituted with C₁₋₆ alkyl or C₆₋₁₀ aryl-C₁₋₄ alkyl and (vii) a halogen atom],
(2) tetrazolyl, 4,5-dihydro-5-oxo-1,2,4-oxadiazolyl, 4,5-dihydro-5-thioxo-1,2,4-oxadiazolyl, 2,3-dihydro-3-oxo-1,2,4-oxadiazolyl, 2,3-dihydro-3-thioxo-1,2,4-oxadiazolyl, 3,5-dioxo-1,2,4-oxadiazolydinyl, 4,5-dihydro-5-oxo-isoxazolyl, 4,5-dihydro-5-thioxo-isoxazolyl, 2,3-dihydro-2-oxo-1,3,4-oxadiazolyl, 2,3-dihydro-3-oxo-1,2,4-tetrazolyl or 2,3-dihydro-3-thioxo-1,2,4-tetrazolyl group, or
(3) an acyl group selected from (i) a C₂₋₇ alkanoyl group which may be substituted with 1 to 2 halogen atoms, and (ii) a C₆₋₁₀ arylsulfonyl group optionally substituted with 1 to 4 substituents selected from C₁₋₃ alkyl, C₁₋₃ alkoxy and a halogen atom, a C₁₋₄ alkylsulfonyl group or a C₆₋₁₀ aryl-C₁₋₄ alkylsulfonyl group,
or R_{2b} and R_{3b} may be combined together with the adjacent nitrogen atom to form 5- or 6-membered ring derived from piperidine, piperazine, pyrrolidine, 2-oxopiperazine, 2,6-dioxopiperazine, morpholine or thiomorpholine [wherein, the 5-membered or 6-membered ring is optionally substituted with 1 to 4 substituent selected from (A) hydroxy group optionally substituted with C₁₋₃ alkyl or C₂₋₇ alkanoyl, (B) carboxyl group optionally esterified with C₁₋₆ alkyl or C₆₋₁₀ aryl-C₁₋₄ alkyl, (C) phosphate group optionally mono- or di-substituted with C₁₋₆ alkyl or C₂₋₇ alkanoyloxy-C₁₋₆ alkyl, (D) a sulfonate group, (E) sulfonamide group optionally substituted with C₁₋₆ alkyl or C₆₋₁₀ aryl-C₁₋₄ alkyl, (F) C₁₋₆ alkyl and C₂₋₅ alkenyl, each of which may be substituted with a carboxyl group optionally esterified with C₁₋₆ alkyl or C₆₋₁₀ aryl-C₁₋₄ alkyl; a phosphate group optionally mono- or di-substituted with C₁₋₆ alkyl or C₂₋₇ alkanoyloxy-C₁₋₆ alkyl; a sulfonate group; sulfonamide group optionally substituted with C₁₋₆ alkyl or C₆₋₁₀ aryl-C₁₋₄ alkyl; a hydroxy group optionally substituted with C₁₋₃ alkyl or C₂₋₇ alkanoyl; a sulfhydryl group optionally alkylated with C₁₋₃ alkyl; a carbamoyl group; phenyl optionally substituted with 1 to 5 substituents selected from a hydroxy group, a halogen atom, an aminosulfonyl and an amino group optionally substituted with C₁₋₃ alkyl; an amino group optionally mono- or di-substituted with C₁₋₃ alkyl; or tetrazolyl, (G) amino group optionally mono- or di-substituted with C₁₋₃ alkyl, (H) a cyclic amino group derived from piperidine, pyrrolidine, morpholine, thiomorpholine, 4-methylpiperazine, 4-benzylpiperazine or 4-phenylpiperazine, (I) a cyano group, (J) a carbamoyl group, (K) an oxo group, (L) a tetrazolyl or 2,5-dihydro-5-oxo-1,2,4-oxadiazolyl group, (M) carbamoyl group optionally substituted with C₆₋₁₀ arylsulfonyl, C₁₋₄ alkylsulfonyl or C₆₋₁₀ aryl-C₁₋₄ alkylsulfonyl, (N) sulfhydryl group optionally alkylated with C₁₋₃ alkyl, and (O) phenyl group which may be substituted with 1 to 5 substituents selected from a hydroxy group, a halogen atom, aminosulfonyl and amino group optionally substituted with C₁₋₃ alkyl];
the compound wherein in a group represented by X_{b}, R_{2b} and R_{3b} may be combined together with the adjacent nitrogen atom of carbamoyl group to form a 5- or 6-membered ring derived from piperidine, piperazine, pyrrolidine, 2-oxopiperazine or 2,6-dioxopiperazine, and the 5- or 6-membered ring may be substituted with C₁₋₆ alkyl group optionally having 1 to 2 substituents selected from (i) carboxyl group optionally esterified with C₁₋₆ alkyl or C₆₋₁₀ aryl-C₁₋₄ alkyl, (ii) phosphate group optionally mono- or di-substituted with C₁₋₆ alkyl or C₂₋₇ alkanoyl-C₁₋₆ alkyl, (iii) a sulfonate group, (iv) sulfonamide group optionally substituted with C₁₋₆ alkyl or C₆₋₁₀ aryl-C₁₋₄ alkyl, (v) hydroxy group optionally alkylated with C₁₋₃ alkyl, (vi) sulfhydryl group optionally alkylated with C₁₋₃ alkyl, (vii) a carbamoyl group, (viii) phenyl group which may be substituted with 1 to 5 substituents selected from a hydroxy group, a halogen atom, aminosulfonyl and amino optionally substituted with C₁₋₃ alkyl, (ix) amino group optionally mono- or di-substituted with C₁₋₃ alkyl, and (x) a tetrazolyl group;
the compound wherein in a group represented by X_{b}, R_{2b} is a hydrogen atom or C₁₋₇ alkyl and R_{3b} is C₁₋₄ alkylsulfonyl; the compound wherein the heterocyclic group represented by X_{b} is tetrazolyl, 4,5-dihydro-5-oxo-1,2,4-oxadiazolyl, 4,5-dihydro-5-thioxo-1,2,4-oxadiazolyl, 2,3-dihydro-3-oxo-1,2,4-oxadiazolyl, 2,3-dihydro-3-thioxo-1,2,4-oxadiazolyl, 3,5-dioxo-1,2,4-oxadiazolydinyl, 4,5-dihydro-5-oxo-isoxazolyl, 4,5-dihydro-5-thioxo-isoxazolyl, 2,3-dihydro-2-oxo-1,3,4-oxadiazolyl, 2,3-dihydro-3-oxo-1,2,4-tetrazolyl or 2,3-dihydro-3-thioxo-1,2,4-tetrazolyl;
the compound wherein R_{1b} is methyl, W is a chlorine atom, R_{b} is C₃₋₆ branched alkyl which is substituted with 1 to 3 substituents selected from a hydroxy group, acetyloxy, propionyloxy, tert-butoxycarbonyloxy, palmitoyloxy, dimethylaminoacetyloxy and 2-aminopropionyloxy, and X_{b} is a group represented by the formula:

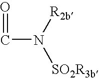

wherein, R_{2b′} represents a hydrogen atom or C₁₋₇ alkyl and R_{3b′} represents C₁₋₄ alkyl;
the compound wherein R_{1b} is methyl, W is a chlorine atom, R_{b} is C₃₋₆ branched alkyl which is substituted with 1 to 3 substituents selected from a hydroxy group, acetyloxy, propionyloxy, tert-butoxycarbonyloxy, palmitoyloxy, dimethylaminoacetyloxy and 2-aminopropionyloxy, and X_{b} is a group represented by the formula:

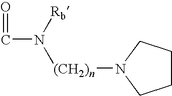

wherein, R_{b′} represents a hydrogen atom or C₁₋₇ alkyl, and n represents an integer of 1 to 5;
the compound wherein R_{1b} is methyl, W is a chlorine atom, R_{b} is C₃₋₆ branched alkyl which is substituted with 1 to 3 substituents selected from a hydroxy group, acetyloxy, propionyloxy, tert-butoxycarbonyloxy, palmitoyloxy, dimethylaminoacetyloxy and 2-aminopropionyloxy, and X_{b} is a group represented by the formula:

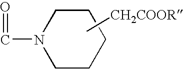

wherein, R″ represents a hydrogen atom or C₁₋₄ alkyl; the compound wherein R_{1b} is methyl, W is a chlorine atom, R_{b} is C₃₋₆ branched alkyl which is substituted with 1 to 3 substituents selected from a hydroxy group, acetyloxy, propionyloxy, tert-butoxycarbonyloxy, palmitoyloxy, dimethylaminoacetyloxy and 2-aminopropionyloxy, and X_{b} is tetrazolyl;
the compound wherein R_{b} is lower alkyl optionally substituted with 1 or 2 hydroxy groups, and X_{b} is (1) carbamoyl group optionally substituted with a hydrocarbon group selected from (a) C₁₋₇ alkyl, (b) C₃₋₇ cycloalkyl, (c) C₂₋₆ alkenyl, (d) C₆₋₁₀ aryl and (e) C₇₋₁₄ arylalkyl [wherein, (a) C₁₋₇ alkyl, (b) C₃₋₇ cycloalkyl and (c) C₂₋₆ alkenyl may respectively have 1 to 4 substituents selected from (i) carboxyl group optionally esterified with C₁₋₆ alkyl or C₇₋₁₀ arylalkyl, (ii) a phosphate group, (iii) a sulfonate group, (iv) sulfonamide group optionally substituted with C₁₋₆ alkyl or C₇₋₁₀ arylalkyl, (v) hydroxy group optionally alkylated with C₁₋₃ alkyl, (vi) sulfhydryl group optionally alkylated with C₁₋₃ alkyl, (vii) a carbamoyl group, (viii) an phenyl group optionally substituted with substituents selected from a hydroxy group, a chlorine atom, a fluorine atom, aminosulfonyl and amino group optionally mono- or di-substituted with C₁₋₃ alkyl, (ix) amino group optionally mono- or di-substituted with C₁₋₃ alkyl, and (x) cyclic amino group derived from piperidine, pyrrolidine, morpholine, thiomorpholine, piperazine, 4-methylpiperazine, 4-benzylpiperazine, or 4-phenylpiperazine, which may be substituted with C₁₋₃ alkyl, benzyl or phenyl and (xi) 5- or 6-membered aromatic heterocyclic group derived from pyridine, imidazole, indole or tetrazole, and (d) C₆₋₁₀ aryl and (e) C₇₋₁₄ arylalkyl may respectively have 1 to 4 substituents selected from (i) carboxyl group optionally esterified with C₁₋₄ alkyl, (ii) a phosphate group, (iii) a sulfonate group, (iv) sulfonamide group optionally substituted with C₁₋₆ alkyl or C₇₋₁₀ arylalkyl, (v) C₁₋₃ alkyl group optionally substituted with carboxyl group which may be esterified with C₁₋₄ alkyl, a phosphate group, a sulfonate group, or sulfonamide group optionally substituted with C₁₋₆ alkyl or C₇₋₁₀ arylalkyl, and (iv) a halogen atom],
(2) tetrazolyl, 4,5-dihydro-5-oxo-1,2,4-oxadiazolyl, 4,5-dihydro-5-thioxo-1,2,4-oxadiazolyl, 2,3-dihydro-3-oxo-1,2,4-oxadiazolyl, 2,3-dihydro-3-thioxo-1,2,4-oxadiazolyl, 3,5-dioxo-1,2,4-oxadiazolydinyl, 4,5-dihydro-5-oxo-isoxazolyl, 4,5-dihydro-5-thioxo-isoxazolyl, 2,3-dihydro-2-oxo-1,3,4-oxadiazolyl, 2,3-dihydro-3-oxo-1,2,4-tetrazolyl or 2,3-dihydro-3-thioxo-1,2,4-tetrazolyl group,
(3) carbamoyl group optionally substituted with an acyl group selected from (i) a C₂₋₇ alkanoyl group optionally substituted with 1 to 2 halogen atoms, and
(ii) a C₆₋₁₀ arylsulfonyl group, a C₁₋₄ alkylsulfonyl group or a C₇₋₁₄ arylalkylsulfonyl group, each of which may be substituted with 1 to 4 substituents selected from C₁₋₃ alkyl, C₁₋₃ alkoxy and a halogen atom, or
(4) a cyclic amino carbonyl group derived from piperidine, piperazine, pyrrolidine, 2-oxopiperazine, 2,6-dioxopiperazine, morpholine and thiomorpholine [wherein, the cyclic amino carbonyl group may have 1 to 4 substituents selected from (A) a hydroxy group, (B) carboxyl group optionally esterified with C₁₋₄ alkyl, (C) a phosphate group, (D) a sulfonate group, (E) sulfonamide group optionally substituted with C₁₋₆ alkyl or C₇₋₁₀ arylalkyl, (F) C₁₋₃ alkyl or C₂₋₅ alkenyl, each of which may be substituted with the above-mentioned (A), (B), (C), (D) or (E), (G) amino group optionally mono- or di-substituted with C₁₋₃ alkyl, (H) a cyclic amino group derived from piperidine, pyrrolidine, morpholine, thiomorpholine, 4-methylpiperazine, 4-benzylpiperazine or 4-phenylpiperazine, (I) a cyano group, (J) a carbamoyl group, (K) oxo, (L) C₁₋₃ alkoxy, (M) a heterocyclic group derived from tetrazolyl or 2,5-dihydro-5-oxo-1,2,4-oxadiazolyl, and (N) carbamoyl group optionally substituted with C₆₋₁₀ arylsulfonyl, C₁₋₄ alkylsulfonyl or C₇₋₁₄ arylalkylsulfonyl];
the compound wherein R_{b} is a 2,2-dimethyl-3-hydroxypropyl group; or the like.

In the aforementioned formula, examples of the lower alkyl group represented by R_{b} include C₁₋₆ alkyl such as methyl, ethyl, n-propyl, isopropyl, n-butyl, isobutyl, n-pentyl, isopentyl, neopentyl and hexyl. Among these, a C₃₋₆ alkyl group is preferred, and a C₄₋₅ alkyl group is more preferred. In particular, a branched C₄₋₅ alkyl group such as isobutyl and neopentyl is preferred.

Examples of the substituent of the lower alkyl represented by R_{b} include hydroxy group optionally substituted with C₂₋₂₀ alkanoyl or C₁₋₇ alkyl, and the like. Examples of these substituents include a hydroxy group, acetyloxy, propionyloxy, tert-butoxycarbonyloxy, palmitoyloxy, dimethylaminoacetyloxy and 2-aminopropionyloxy.

One to three of these substituents may be present at their substitutable positions.

In addition, examples of the optionally substituted lower alkyl represented by R_{b} include 2,2-dimethyl-3-hydroxypropyl, 3-hydroxy-2-hydroxymethyl-2-methylpropyl, 3-acetoxy-2,2-dimethylpropyl, 3-acetoxy-2-hydroxymethyl-2-methyl-propyl and 3-acetoxy-2-acetoxymethyl-2-methylpropyl.

The optionally substituted carbamoyl group represented by X_{b} includes a group represented by the formula:

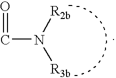

Examples of the “optionally substituted hydrocarbon group” represented by R_{2b} and R_{3b} include an optionally substituted C₁₋₇ linear or branched alkyl group (e.g., methyl, ethyl, n-propyl, isopropyl, n-butyl, isobutyl, 1,1-dimethylethyl, n-pentyl, 3-methylbutyl, 2-methylbutyl, 1-methylbutyl, 1-ethylpropyl, n-hexyl, 4-methylpentyl, 3-methylpentyl, 2-methylpentyl, 2-ethylbutyl, 1-ethylbutyl, neopentyl, hexyl, heptyl), an optionally substituted C₃₋₇ cycloalkyl group (e.g., cyclopropyl, cyclobutyl, cyclopentyl, cyclohexyl, cyclohexylmethyl, etc.), an optionally substituted C₂₋₆ linear or branched alkenyl group (e.g., vinyl, allyl, isopropenyl, 2-methylallyl, 1-propenyl, 2-methyl-1-propenyl, 2-methyl-2-propenyl, 1-butenyl, 2-butenyl, 3-butenyl, 2-ethyl-1-butenyl, 2-methyl-2-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl etc.), an optionally substituted C₆₋₁₀ aryl group (e.g., phenyl and naphthyl group) and an optionally substituted C₇₋₁₄ arylalkyl group (e.g., benzyl, phenethyl and naphthylmethyl).

Examples of the substituent of the “optionally substituted C₁₋₇ linear or branched alkyl group, optionally substituted C₃₋₇ cycloalkyl group and optionally substituted C₂₋₆ linear or branched alkenyl group” include carboxyl group optionally esterified with C₁₋₆ alkyl group or C₆₋₁₀ aryl-C₁₋₄ alkyl group (e.g., methyl, ethyl, propyl, isopropyl, butyl, tert-butyl, phenyl, benzyl, etc.), phosphate group optionally mono- or di-substituted with C₁₋₆ alkyl group (e.g., methyl, ethyl, n-propyl, isopropyl, n-butyl, isobutyl, n-pentyl, isopentyl, neopentyl, hexyl, etc.) or C₂₋₇ alkanoyloxy-C₁₋₆ alkyl such as acetyloxymethyl or pivaloyloxymethyl group, a sulfonate group, sulfonamide group optionally substituted with C₁₋₆ alkyl group or C₆₋₁₀ aryl-C₁₋₄ alkyl group (e.g., methyl, ethyl, propyl, isopropyl, butyl, tert-butyl, benzyl, etc.), hydroxy group and sulfhydryl group, each optionally alkylated with C₁₋₃ alkyl group (e.g., methyl, ethyl, propyl, etc.), a carbamoyl group, phenyl group optionally substituted with 1 to 5 substituents [e.g., a hydroxy group, chlorine, fluorine, an aminosulfonyl group or amino group optionally substituted with C₁₋₃ alkyl group (e.g., methyl, ethyl, propyl, etc.)], an amino group optionally mono- or di-substituted with C₁₋₃ alkyl group (e.g., methyl, ethyl, propyl, etc.), a cyclic amino group (e.g., 5- or 6-membered cyclic amino group derived from cyclic amine such as piperidine, pyrrolidine, morpholine, thiomorpholine, piperazine, 4-methylpiperazine, 4-benzylpiperazine, 4-phenylpiperazine, 1,2,3,4-tetrahydroisoquinoline and phthalimide, which may be substituted with a C₁₋₃ alkyl group, benzyl, phenyl or the like, and further optionally contains an oxygen atom or a sulfur atom as a ring constituent atom) and a 5- to 6-membered aromatic heterocyclic ring containing 1 to 4 hetero atoms selected from N, O and S (e.g., pyridine, imidazole, indole, tetrazole, etc).

In addition, examples of the substituent of C₆₋₁₀ aryl group and C₆₋₁₀ aryl-C₁₋₄ alkyl group as the substituent of an optionally substituted amino group which composes a carbamoyl group of the “optionally substituted carbamoyl group” represented by X_{b} include carboxyl group optionally esterified with C₁₋₄ alkyl group (methyl, ethyl, propyl, tert-butyl group, etc.), phosphate group optionally mono- or di-substituted with C₁₋₆ alkyl group (methyl, ethyl, n-propyl, isopropyl, n-butyl, isobutyl, n-pentyl, isopentyl, neopentyl, hexyl) or C₂₋₇ alkanoyloxy-C₁₋₆ alkyl such as pivaloyloxymethyl group and acetyloxymethyl group, a sulfonate group, C₁₋₄ alkylsulfonyl, C₆₋₁₀ arylsulfonyl or C₆₋₁₀ aryl-C₁₋₄ alkylsulfonyl, sulfonamide group optionally substituted with a C₁₋₆ alkyl group or a C₆₋₁₀ aryl-C₁₋₄ alkyl group (e.g., methyl, ethyl, propyl, isopropyl, butyl, tert-butyl, benzyl, etc.) and carboxyl group optionally esterified with a C₁₋₄ alkyl group, phosphate group optionally mono- or di-substituted with a C₁₋₆ alkyl group such as methyl, ethyl, n-propyl, isopropyl, n-butyl, isobutyl, n-pentyl, isopentyl, neopentyl and hexyl or C₂₋₇ alkanoyloxy-C₁₋₆ alkyl group such as pivaloyloxymethyl group, C₁₋₃ alkyl group (e.g., methyl, ethyl, propyl and isopropyl) optionally substituted with a sulfonate group and sulfonamide group optionally substituted with C₁₋₆ alkyl or a C₆₋₁₀ aryl-C₁₋₄ alkyl group, and a halogen atom (e.g., fluorine and chlorine), and the like.

The “hydrocarbon group” may have 1 to 5 substituents at a substitutable position.

The “optionally substituted heterocyclic group” represented by R_{2b} and R_{3b} may have 1 to 2 (preferably one) of substituents such as oxo group and thioxo group, and preferred is a heterocyclic group having a hydrogen atom which can be deprotonated. Such heterocyclic group is preferably a 5- to 6-membered heterocyclic group containing 1 to 4, preferably 2 to 3 hetero atoms selected from S, O and N. Specific examples include tetrazolyl, 4,5-dihydro-5-oxo-1,2,4-oxadiazolyl, 4,5-dihydro-5-thioxo-1,2,4-oxadiazolyl, 2,3-dihydro-3-oxo-1,2,4-oxadiazolyl, 2,3-dihydro-3-thioxo-1,2,4-oxadiazolyl, 3,5-dioxo-1,2,4-oxadiazolydinyl, 4,5-dihydro-5-oxo-isoxazolyl, 4,5-dihydro-5-thioxo-isoxazolyl, 2,3-dihydro-2-oxo-1,3,4-oxadiazolyl, 2,3-dihydro-3-oxo-1,2,4-tetrazolyl and 2,3-dihydro-3-thioxo-1,2,4-tetrazolyl. Among these, tetrazolyl group is preferred.

Examples of the “acyl group” represented by R_{2b} and R_{3b} include a carboxylic acid acyl group derived from carboxylic acid (e.g., C₂₋₇ carboxylic acid acyl group such as acetyl, propionyl, butyryl, benzoyl, etc.) and C₆₋₁₀ arylsulfonyl group, C₁₋₄ alkylsulfonyl group and C₆₋₁₀ aryl-C₁₋₄ alkylsulfonyl group, each of which may have a substituent (e.g., methylsulfonyl, ethylsulfonyl, phenylsulfonyl, naphthylsulfonyl, phenylmethylsulfonyl, phenylethylsulfonyl, naphthylmethylsulfonyl, naphthylethylsulfonyl, etc.). The substituents of aryl, alkyl and arylalkylsulfonyl group include C₁₋₃ alkyl (e.g., methyl, ethyl, propyl, etc.), C₁₋₃ alkoxy (e.g., methoxy, ethoxy, propoxy, etc.), a halogen atom (e.g., chlorine, fluorine, bromine) and the like, and 1 to 4, preferably 1 to 2, thereof may be present at a substitutable position.

The aforementioned carboxylic acid acyl group may have 1 to 2 halogen atoms (e.g., chlorine, fluorine, bromine) as a substituent.

Examples of the cyclic amino group optionally substituted with C₁₋₃ alkyl, C₂₋₇ alkanoyl or the like, which is formed by combining R_{2b} and R_{3b} together with the adjacent nitrogen atom of carbamoyl group, include a group derived from 5- or 6-membered cyclic amine such as piperazine, piperidine, pyrrolidine, piperazin-2-one, piperazine-2,6-dione, morpholine and thiomorpholine, and said cyclic amine may further contain 1 to 3 hetero atoms selected from a nitrogen atom, a sulfur atom and an oxygen atom as ring constituent atom. Such cyclic amino group may have 1 to 4, preferably 1 to 2 substituents. Examples of the substituents include hydroxy group optionally substituted with C₁₋₃ alkyl group or C₂₋₇ alkanoyl, carboxyl group optionally esterified with C₁₋₄ alkyl group (methyl, ethyl, propyl, tert-butyl, etc.) and C₇₋₁₀ arylalkyl, phosphate group optionally mono- or di-substituted with C₁₋₆ alkyl or C₂₋₇ alkanoyloxy-C₁₋₆ alkyl group (acetyloxymethyl group, pivaloyloxymethyl group), a sulfonate group, and sulfonamide group optionally substituted with C₁₋₆ alkyl or C₆₋₁₀ aryl-C₁₋₄ alkyl group (e.g., methyl, ethyl, propyl, isopropyl, butyl, tert-butyl, benzyl, etc.), C₁₋₆ alkyl and C₂₋₅ alkenyl, each of which may be substituted with “carboxyl group optionally esterified with C₁₋₆ alkyl or C₆₋₁₀ aryl-C₁₋₄ alkyl, phosphate group optionally mono- or di-substituted with C₁₋₆ alkyl or C₂₋₇ alkanoyloxy-C₁₋₆ alkyl group (e.g., acetyloxymethyl group, pivaloyloxymethyl group, etc.), a sulfonate group, sulfonamide group optionally substituted with C₁₋₆ alkyl or C₆₋₁₀ aryl-C₁₋₄ alkyl group, hydroxy group optionally substituted with C₁₋₃ alkyl or C₂₋₇ alkanoyl, sulfhydryl group optionally alkylated with C₁₋₃ alkyl, a carbamoyl group, phenyl group optionally substituted with 1 to 5 substituents (e.g., a hydroxy group, a halogen atom, aminosulfonyl, amino group optionally substituted with C₁₋₃ alkyl, etc.), amino group optionally mono- or di-substituted with C₁₋₃ alkyl or tetrazolyl group”, amino group optionally mono- or di-substituted with C₁₋₃ alkyl (e.g., methyl, ethyl, propyl, etc.), a cyclic amino group (a group derived from 5- or 6-membered cyclic amine which may contain additional hetero atoms selected from a nitrogen atom, a sulfur atom and an oxygen as ring constituent atom, and which may be substituted with C₁₋₃ alkyl, benzyl, phenyl, or the like, for example, piperidine, pyrrolidine, morpholine, thiomorpholine, 4-methylpiperazine, 4-benzylpiperazine, 4-phenylpiperazine, or the like), a cyano group, a carbamoyl group, an oxo group, C₁₋₃ alkoxy (e.g., methoxy, ethoxy, ethylenedioxy, etc.), heterocyclic group optionally substituted with an oxo group or thioxo group having a hydrogen atom which can be deprotonated as mentioned above (e.g., tetrazolyl, 2,5-dihydro-5-oxo-1,2,4-oxadiazolyl, etc.), C₆₋₁₀ arylsulfonyl, C₆₋₁₀ aryl-C₁₋₄ alkylsulfonyl and C₁₋₄ alkylsulfonyl (e.g., methylsulfonyl, ethylsulfonyl, propylsulfonyl, butylsulfonyl, isopropylsulfonyl, tert-butylsulfonyl, phenylsulfonyl, benzylsulfonyl, etc.) which is exemplified for the substituent of an optionally substituted amino group composing carbamoyl of the “optionally substituted carbamoyl group” represented by X, sulfhydryl group optionally alkylated with C₁₋₃ alkyl, or carbamoyl group substituted with phenyl which may be substituted with 1 to 5 substituents (e.g., a hydroxy group, a halogen atom, an aminosulfonyl and amino group optionally substituted with C₁₋₃ alkyl).

Examples of the optionally substituted carbamoyl group represented by X_{b} include:

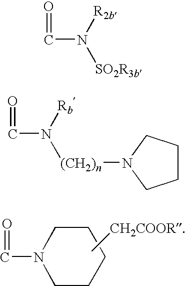

R_{2b′} and R_{b′} include a hydrogen atom and C₁₋₇ alkyl. Hydrogen atom is particularly preferred.

The C₁₋₇ alkyl represented by R_{2b}, R_{2b′} and R_{b}′ includes the same groups as those exemplified with respect to the aforementioned C₁₋₇ alkyl of the “hydrocarbon group”.

R″ includes a hydrogen atom and C₁₋₄ alkyl. Hydrogen atom is particularly preferred.

The C₁₋₄ alkyl represented by R_{3b′} and R″ includes, for example, methyl, ethyl, propyl, isopropyl, n-butyl, tert-butyl, etc.

As for the optionally substituted heterocyclic group having a hydrogen atom which can be deprotonated represented by X_{b}, a nitrogen-containing (preferably containing 1 to 4 nitrogen atoms) 5- to 6-membered heterocyclic ring having Broensted acid-like active proton is preferred, and those containing 1 to 4, preferably 2 to 3 of a nitrogen atom, a sulfur atom and an oxygen atom may be preferred. The substituents thereof include an oxo group and a thioxo group, and 1 to 2, preferably 1, of such substituents may be present. Examples of the “optionally substituted heterocyclic group having a hydrogen atom which can be deprotonated” represented by X are exemplified by those for the “optionally substituted heterocyclic group” as the substituent of the “optionally substituted carbamoyl group” represented by X such as tetrazolyl, 2,5-dihydro-5-oxo-1,2,4-oxadiazolyl and the like.

Examples of the “lower alkyl group” represented by R_{1b} include a C₁₋₆ alkyl group such as methyl, ethyl, n-propyl, isopropyl, n-butyl, tert-butyl, pentyl, hexyl and the like. Among these, C₁₋₃ alkyl group is preferred. In the view of pharmacological activity, methyl group is particularly preferred as R_{1b}.

Examples of the “halogen atom” represented by W include chlorine, fluorine, bromine, iodine atoms. The chlorine atom is particularly preferred.

Examples of the salts of the compound represented by the formula (Ib) include pharmacologically acceptable salts such as inorganic salts such as hydrochloride, hydrobromide, sulfate, nitrate, phosphate and the like; organic salts such as acetate, tartrate, citrate, fumarate, maleate, toluenesulfonate, methanesulfonate and the like; metal salts such as sodium salt, potassium salt, calcium salt, aluminum salt and the like; and salts with base such as triethylamine salt, guanidine salt, ammonium salt, hydrazine salt, quinine salt, cinchonine salt and the like.

In addition, hydrates as well as non-hydrates of the compound represented by the formula (Ib) are included within the scope of the present invention.

The compound represented by the formula (Ib) and salts thereof contains asymmetric carbon atoms at 3- and 5-positions, herein the trans isomer wherein the substituents on 3- and 5-positions are directed in the opposite direction relative to the plane of a 7-membered ring is preferred, and in particular, the isomer wherein the absolute configuration at 3-position is R-configuration and the absolute configuration at 5-position is S-configuration is preferred.

As for the compounds represented by the formula (Ib) or salts thereof, the following compounds are specifically preferred.
N-methanesulfonyl-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetamide,
N-methanesulfonyl-[(3R,5S)-7-chloro-5-(2,3-dimethoxyphenyl)-1-(3-hydroxy-2-hydroxymethyl-2-methylpropyl)-2-oxo-1,2,3,5-tetrahydro-4,1-benzoxazepin-3-yl]acetamide,
N-[2-(pyrrolidin-1-yl)ethyl]-[(3R,5S)-7-chloro-5-(2,3-dimethoxyphenyl)-1-(3-hydroxy-2-hydroxymethyl-2-methylpropyl)-2-oxo-1,2,3,5-tetrahydro-4,1-benzoxazepin-3-yl]acetamide,
N-[2-(pyrrolidin-1-yl)ethyl]-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetamide,
N-methanesulfonyl-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetamide,
N-methanesulfonyl-[(3R,5S)-1-(3-acetoxy-2-acetoxymethyl-2-methylpropyl)-7-chloro-5-(2,3-dimethoxyphenyl)-2-oxo-1,2,3,5-tetrahydro-4,1-benzoxazepin-3-yl]acetamide,
N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid,
N-[[(3R,5S)-1-(3-hydroxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid,
N-[[(3R,5S)-1-(2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid,
N-[[(3R,5S)-1-(3-acetoxy-2-acetoxymethyl-2-methylpropyl)-7-chloro-5-(2,3-dimethoxyphenyl)-2-oxo-1,2,3,5-tetrahydro-4,1-benzoxazepin-3-yl]acetyl]piperidin-4-acetic acid,
ethyl N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetate,
ethyl N-[[(3R,5S)-1-(3-acetoxy-2-acetoxymethyl-2-methylpropyl)-7-chloro-5-(2,3-dimethoxyphenyl)-2-oxo-1,2,3,5-tetrahydro-4,1-benzoxazepin-3-yl]acetyl]piperidine-4-acetate,
(3R,5S)-7-chloro-5-(2,3-dimethoxyphenyl)-1-(3-hydroxy-2,2-dimethylpropyl)-1,2,3,5-tetrahydro-3-[1H (or 3H)-tetrazol-5-yl]methyl-4,1-benzoxapin-2-one,
(3R,5S)-7-chloro-5-(2,3-dimethoxyphenyl)-1-(3-hydroxy-2-hydroxymethyl-2-methylpropyl)-1,2,3,5-tetrahydro-3-[1H (or 3H)-tetrazol-5-yl]methyl-4,1-benzoxazepin-2-one,
(3R,5S)-1-(3-acetoxy-2,2-dimethylpropyl)-7-chloro-5-(2,3-dimethoxyphenyl)-1,2,3,5-tetrahydro-3-[1H (or 3H)-tetrazol-5-yl]methyl-4,1-benzoxazepin-2-one,
(3R,5S)-1-(3-acetoxy-2-acetoxymethyl-2-methylpropyl)-7-chloro-5-(2,3-dimethoxyphenyl)-1,2,3,5-tetrahydro-3-[1H (or 3H)-tetrazol-5-yl]methyl-4,1-benzoxazepin-2-one,
N-[2-(pyrrolidin-1-yl)ethyl]-[(3R,5S)-7-chloro-5-(2,3-dimethoxyphenyl)-1-neopentyl-2-oxo-1,2,3,5-tetrahydro-4,1-benzoxazepin-3-yl]acetamide, and the like.

The compound represented by the formula (Ib) and salts thereof can be prepared according to the methods disclosed in the publications, for example, EP-A-567026, WO95/21834 (PCT application based on Japanese Patent Application No. 6-15531), EP-A-645377 (an application based on Japanese Patent Application No. 6-229159), EP-A-645378 (an application based on Japanese Patent Application No. 6-229160), WO97/10224 and the like, or the methods similar thereto.

As the compound represented by the formula (I), the compound represented by the aforementioned formula (Ic):

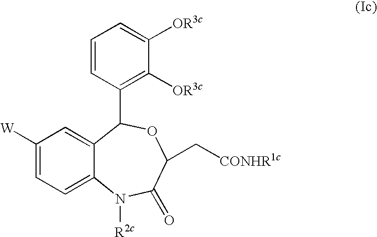

is preferred.

Preferable examples of the compound represented by the formula (Ic) include:

the compound wherein R^{1c} is a 3-carboxypropyl group, a 1-carboxyethyl group, or a C₃₋₆ linear alkyl-sulfonyl group, a (carboxy-C₅₋₇ cycloalkyl)-C₁₋₃ alkyl group, a (carboxyfuryl)-alkyl group, a carboxy-C₆₋₁₀ aryl group, a (carboxy-C₂₋₃ alkyl)-C₆₋₁₀ aryl group or a (carboxy-C₁₋₃ alkyl)-C₇₋₁₄ aralkyl group, each of which may be optionally substituted; the compound wherein R_{1c} is a (carboxy-C₁₋₄ alkyl)-C₆₋₁₀ aryl group which may have a substituent;
the compound wherein R^{1c} is a (carboxy-C₂₋₃ alkyl)-C₆₋₁₀ aryl group which may have a substituent;
the compound wherein R^{1c} is a (carboxy-C₂₋₃ alkyl)-phenyl group which may have a substituent;
the compound wherein R^{1c} is a (carboxyfuryl)-alkyl group which may have a substituent;
the compound wherein R^{2c} is a C₃₋₆ alkyl group which have alkanoyloxy group and/or a hydroxy group;
the compound wherein R^{2c} is a C₃₋₆ alkyl group which may have 1 to 3 substituents selected from hydroxy group, acetoxy, propionyloxy, tert-butoxycarbonyloxy and palmitoyloxy;
the compound wherein R^{2c} is 2,2-dimethylpropyl, 3-hydroxy-2,2-dimethylpropyl or 3-acetoxy-2,2-dimethylpropyl;
the compound wherein R^{3c} is methyl group;
the compound wherein W is chlorine atom;
the compound having R-configuration at 3-position, and S-configuration at 5-position; and the like.

In the aforementioned formula, R^{1c} represents an optionally substituted 1-carboxyethyl group, an optionally substituted carboxy-C₃₋₆ linear alkyl group, an optionally substituted C₃₋₆ linear alkyl-sulfonyl group, an optionally substituted (carboxy-C₅₋₇ cycloalkyl)-C₁₋₃ alkyl group, or a group represented by formula —X^{1c}—X^{2c}—Ar—X^{3c}—X^{4c}—COOH (wherein X^{1c} and X^{4c} respectively represents a bond or an optionally substituted C₁₋₄ alkylene group, X^{2c} and X^{3c} respectively represents a bond, —O— or —S—, and Ar represents an optionally substituted divalent aromatic ring group. Provided that, when X^{1c} is a bond, X^{2c} represents a bond, and when X^{4c} is a bond, X represents a bond).

Examples of the C₃₋₆ linear alkyl group in the optionally substituted carboxy-C₃₋₆ linear alkyl group represented by R_{1c} include n-propyl, n-butyl, n-pentyl, n-hexyl. Among these, n-propyl and n-butyl are preferred, and n-propyl is more preferred.

Examples of the C₃₋₆ linear alkyl group in the optionally substituted C₃₋₆ linear alkyl-sulfonyl group represented by R_{1c} include n-propyl, n-butyl, n-pentyl, n-hexyl. Among these, n-propyl and n-butyl are preferred, and n-propyl is more preferred.

Examples of the C₅₋₇ cycloalkyl group in the optionally substituted (carboxy-C₅₋₇ cycloalkyl)-C₁₋₃ alkyl group represented by R^{1c} include cyclopentyl, cyclohexyl and cycloheptyl. Among these, cyclopentyl and cyclohexyl are preferred, and cyclohexyl is more preferred.

Examples of the C₁₋₃ alkyl group in the optionally substituted (carboxy-C₅₋₇ cycloalkyl)-C₁₋₃ alkyl group represented by R^{1c} include methyl, ethyl, n-propyl and isopropyl. Among these, methyl and ethyl are preferred, and methyl is more preferred.

In the group represented by formula —X^{1c}—X^{2c}—Ar—X^{3c}—X^{4c}—COOH for R^{1c}, examples of the “C₁₋₄ alkylene group” in the “optionally substituted C₁₋₄ alkylene group” represented by X^{1c} and X^{4c} include methylene, dimethylene, trimethylene, tetramethylene, and C₁₋₃ alkylene group is preferred, and among them, the linear one may be preferably used.

Examples of the “divalent aromatic ring group” in the “optionally substituted divalent aromatic ring group” represented by Ar include a divalent aromatic hydrocarbon group, a divalent aromatic heterocyclic group, and the like.

Herein, examples of the divalent aromatic hydrocarbon group include a group formed by removing one hydrogen atom from C₆₋₁₀ aryl group (e.g., phenyl, naphthyl, etc.), and phenylene is preferably used as a divalent aromatic hydrocarbon group.

Examples of the divalent aromatic heterocyclic group include a group formed by removing one hydrogen atom from an aromatic heterocyclic group containing at least 1 (preferably 1 to 4, more preferably 1 to 2) of 1 to 3 (preferably 1 to 2) kinds of hetero atoms selected from an oxygen atom, a sulfur atom and a nitrogen atom as ring-system constituent atoms (ring atom).

Herein, examples of the aromatic heterocyclic group include a 5- to 6-membered aromatic monocyclic heterocyclic group such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl and triazinyl (preferably, furyl, thienyl, pyrrolyl, imidazolyl, thiazolyl, pyridyl, etc.) and an 8- to 12-membered aromatic fused heterocyclic group such as benzofuranyl, isobenzofuranyl, benzo[b]thienyl, indolyl, isoindolyl, 1H-indazolyl, benzimidazolyl, benzoxazolyl, 1,2-benzoisoxazolyl, benzothiazolyl, benzopyranyl, 1,2-benzoisothiazolyl, 1H-benzotriazolyl, quinolyl, isoquinolyl, cinnolyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, phenathridinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl and 1,2,4-triazolo[4,3-b]pyridazinyl (preferably, a heterocyclic group in which the aforementioned 5- to 6-membered aromatic monocyclic heterocyclic group is fused with a benzene ring, or a heterocyclic group in which two of the same or different 5- to 6-membered aromatic monocyclic heterocyclic groups mentioned above are fused, and more preferably, a heterocyclic group in which the aforementioned 5- to 6-membered aromatic monocyclic heterocyclic group is fused with a benzene ring).

Examples of the substituent which may be possessed by the “C₁₋₄ alkylene group” in the “optionally substituted C₁₋₄ alkylene group” represented by X^{1c} and X^{4c}; and the “divalent aromatic ring group” in the “optionally substituted divalent aromatic ring group” include: (i) carboxyl group optionally esterified with a C₁₋₆ alkyl group or a C₆₋₁₀ aryl-C₁₋₄ alkyl group (e.g., methyl, ethyl, propyl, isopropyl, butyl, tert-butyl, phenyl, benzyl, etc.), (ii) phosphate group optionally mono- or di-substituted with C₁₋₆ alkyl (e.g., methyl, ethyl, n-propyl, isopropyl, n-butyl, isobutyl, n-pentyl, isopentyl, neopentyl, hexyl, etc.) or C₂₋₇ alkanoyloxy-C₁₋₆ alkyl such as acetoxymethyl and pivaloyloxymethyl group, (iii) a sulfonate group, (iv) sulfonamide group optionally substituted with a C₁₋₆ alkyl group or a C₆₋₁₀ aryl-C₁₋₄ alkyl group (e.g., methyl, ethyl, propyl, isopropyl, butyl, tert-butyl, benzyl, etc.), (v) hydroxy group and a sulfhydryl group, each of which may be alkylated with a C₁₋₃ alkyl group (e.g., methyl, ethyl, propyl, etc.), (vi) a carbamoyl group, (vii) phenyl group which may be substituted with 1 to 5 substituents [e.g. a hydroxy group, chlorine, fluorine, aminosulfonyl group, and amino group optionally substituted with C₁₋₃ alkyl group (e.g. methyl, ethyl, propyl, etc.)], and may be bound via O or S, (viii) amino group optionally mono- or di-substituted with a C₁₋₃ alkyl group (e.g. methyl, ethyl, propyl, etc.), (ix) cyclic amino group optionally substituted with 1 to 3 of C₁₋₃ alkyl group (e.g., methyl, ethyl, etc.), benzyl, phenyl and the like (e.g., a 5- to 6-membered cyclic amino group which may contain an oxygen atom or a sulfur atom as ring constituent atoms in addition to a nitrogen atom of the cyclic amino group derived (by removing one hydrogen atom) from a cyclic amine such as piperidine, pyrrolidine, morpholine, thiomorpholine, piperazine, 4-methylpiperazine, 4-benzylpiperazine, 4-phenylpiperazine, 1,2,3,4-tetrahydroisoquinoline, phthalimide, etc.), (x) a 5- to 6-membered aromatic heterocyclic group which may contain 1 to 4 hetero atoms selected from N, O and S, and may be bound via O or S (e.g., pyridyl, imidazolyl, indolyl, tetrazolyl, etc.), (xi) a halogen atom (e.g., chlorine, fluorine, bromine, iodine, etc.), (xii) an C₁₋₄ alkyl group (e.g., methyl, ethyl, propyl, isopropyl, butyl, tert-butyl, etc.), C₁₋₄ alkoxy group (e.g., methoxy, ethoxy, propoxy, isopropoxy, butoxy, t-butoxy, etc.) or C₁₋₄ alkylthio group (e.g., methylthio, ethylthio, propylthio, isopropylthio, butylthio, tert-butylthio, etc.), each of which may be substituted with a substituent selected from C₁₋₄ alkoxy group, C₁₋₄ alkylthio group, carboxyl group and phenyl group, (xiii) a C₅₋₇ cycloalkyl group (e.g., cyclopentyl, cyclohexyl, cycloheptyl, etc.), and (xiv) a C₁₋₇ alkanoyloxy (e.g., formyloxy, acetoxy, propionyloxy, butyryloxy, t-butoxycarbonyloxy, isobutyryloxy, valeryloxy, pivaloyloxy, etc.). 1 to 6, preferably 1 to 3 of these substituents may be present at a substitutable position. In addition, two substituents may be combined to form C₃₋₆ alkylene, C₃₋₆ alkyleneoxy, C₃₋₆ alkylenedioxy or the like, for example, when two adjacent substituents on a phenyl group are combined to form C₄ alkylene, tetrahydronaphthalene group is formed.

Specific examples of the group represented by formula —X^{1c}—X^{2c}—Ar—X —X^{4c}—COOH in R^{1c} include an optionally substituted (carboxy-heteroaryl)-C₁₋₄ alkyl group [preferably, an optionally substituted (carboxy-furyl)-C₁₋₄ alkyl group], an optionally substituted (carboxy-C₆₋₁₀ aryl)-C₁₋₄ alkyl group, an optionally substituted carboxy-heteroaryl group, an optionally substituted carboxy-C₆₋₁₀ aryl group, an optionally substituted (carboxy-C₁₋₄ alkyl)-heteroaryl group, an optionally substituted (carboxy-C₁₋₄ alkyl)-C₆₋₁₀ aryl group [preferably, a (carboxy-C₂₋₃ alkyl)-C₆₋₁₀ aryl group], an optionally substituted (carboxy-C₁₋₄ alkyl)-heteroaryl-C₁₋₄ alkyl group, an optionally substituted (carboxy-C₁₋₄ alkyl)-C₇₋₁₄ aralkyl group [preferably, an optionally substituted (carboxy-C₁₋₃ alkyl)-C₇₋₁₄ aralkyl group], an optionally substituted (carboxy-C₁₋₄ alkoxy)-C₆₋₁₀ aryl group, an optionally substituted (carboxy-C₁₋₄ alkoxy)-C₆₋₁₀ aryl-C₁₋₄ alkyl group, an optionally substituted (carboxy-C₁₋₄ alkyl)-C₆₋₁₀ aryloxy-C₁₋₄ alkyl group, an optionally substituted (carboxy-C₆₋₁₀ aryloxy)-C₁₋₄ alkyl group and an optionally substituted (carboxy-C₁₋₄ alkylthio)-heteroaryl group.

Herein, the same group as the aforementioned “aromatic heterocyclic group” may be exemplified for heteroaryl, and the heteroaryl may have the same substituent as the substituent which the aforementioned “aromatic heterocyclic group” may have. In addition, examples of C₆₋₁₀ aryl include phenyl, naphthyl, azulenyl, and phenyl is preferably used. The C₆₋₁₀ aryl may have the same substituent as the substituent which the aforementioned “aromatic heterocyclic group” may have.

Examples of the alkyl group in the optionally substituted (carboxyfuryl)-C₁₋₄ alkyl group represented by R¹ include C₁₋₄ linear or branched alkyl group such as methyl, ethyl, n-propyl, isopropyl, n-butyl, isobutyl, 1,1-dimethylethyl, and the like. Among these, a C₁₋₄ alkyl group such as methyl, ethyl, n-propyl, isopropyl and n-butyl are preferred, and methyl, ethyl and n-propyl are more preferred. Examples of the carboxyfuryl group include 3-carboxy-2-furyl, 4-carboxy-2-furyl, 2-carboxy-3-furyl, 2-carboxy-5-furyl and the like. Among these, 3-carboxy-2-furyl and 4-carboxy-2-furyl are preferred, and 3-carboxy-2-furyl is more preferred.

Examples of the C₂₋₃ alkyl group in the optionally substituted (carboxy-C₂₋₃ alkyl)-C₆₋₁₀ aryl group represented by R_{1c} include ethyl, n-propyl and isopropyl, and ethyl and n-propyl are preferred. Examples of the C₆₋₁₀ aryl group include phenyl, naphthyl and azulenyl, and phenyl is preferred.

Examples of the C₁₋₃ alkyl group in the optionally substituted (carboxy-C₁₋₃ alkyl)-C₇₋₁₄ aralkyl group represented by R^{1c} include methyl, ethyl, n-propyl and isopropyl, and methyl and ethyl are preferred, and ethyl is particularly preferred. Examples of a C₇₋₁₄ aralkyl group (a C₆₋₁₀ aryl-C₁₋₄ alkyl group) include phenylmethyl, 1-phenylethyl, 2-phenylethyl, 3-phenylpropyl, 2-phenylpropyl, 4-phenylbutyl, (1-naphthyl)methyl, (2-naphthyl)methyl, 1-(1-naphthyl)ethyl, 1-(2-naphthyl)ethyl, 3-(1-naphthyl)propyl, 3-(1-naphthyl)propyl, 4-(1-naphthyl)butyl and 4-(2-naphthyl)butyl, and phenylmethyl, 1-phenylethyl, 3-phenylpropyl, (1-naphthyl)methyl, (2-naphthyl)methyl, (1-naphthyl)ethyl and (2-naphthyl)ethyl are preferred, and phenylmethyl and 2-phenylethyl are particularly preferred.

As for the substituent in case that each group represented by R^{1c} has a substituent, the same as in the substituent which the “divalent aromatic ring group” in the “optionally substituted divalent aromatic ring group” represented by Ar may have may be exemplified, and 1 to 6, preferably 1 to 3 of these substituents can be present at substitutable positions. In addition, in each group represented by R^{1c}, it is preferable that the carboxylic portion is unsubstituted, and an arbitrary portion other than the carboxylic portion may have a substitutable substituent at a substitutable position.

As for R^{1c}, 3-carboxypropyl group, 1-carboxyethyl group, or a C₃₋₆ linear alkyl-sulfonyl group, a (carboxy-C₅₋₇ cycloalkyl)-C₁₋₃ alkyl group, a (carboxyfuryl)-alkyl group, a carboxy-C₆₋₁₀ aryl group, a (carboxy-C₁₋₄ alkyl)-C₆₋₁₀ aryl group [preferably, a (carboxy-C₂₋₃ alkyl)-C₆₋₁₀ aryl group], and a (carboxy-C₁₋₃ alkyl)-C₇₋₁₄ aralkyl group, each of which may have a substituent, and the like are preferred, an optionally substituted (carboxy-C₁₋₄ alkyl)-C₆₋₁₀ aryl group is preferred, and an optionally substituted (carboxy-C₂₋₃ alkyl)-C₆₋₁₀ aryl group is more preferred. In particular, an optionally substituted (carboxy-C₂₋₃ alkyl)-phenyl group is preferred.

Examples of the C₃₋₆ alkyl group in the C₃₋₆ alkyl group optionally substituted with an alkanoyloxy group or a hydroxy group represented by R^{2c} include n-propyl, isopropyl, 1,1-dimethylethyl, n-butyl, isobutyl, n-pentyl, 2,2-dimethylpropyl, isopentyl, n-hexyl, isohexyl and the like. Among these, isopropyl, 1,1-dimethylethyl, n-butyl, isobutyl, 2,2-dimethylpropyl and isohexyl are preferred, and 2,2-dimethylpropyl is particularly preferred.

Examples of the alkanoyloxy group in the C₃₋₆ alkyl group optionally substituted with an alkanoyloxy group or a hydroxy group represented by R include a C₁₋₂₀ alkanoyloxy group such as formyloxy, acetoxy, propionyloxy, butyryloxy, tert-butoxycarbonyloxy, isobutyryloxy, valeryloxy, pivaloyloxy, lauryloxy, palmitoyloxy, stearoyloxy (preferably a C₁₋₇ alkanoyloxy group, etc.). Among these, acetoxy, propionyloxy, tert-butoxycarbonyloxy and palmitoyloxy are preferred, and acetoxy is particularly preferred. 1 to 3 of the alkanoyloxy groups or the hydroxy groups may substitute at a substitutable position.

Preferable examples of C₃₋₆ alkyl group optionally substituted with an alkanoyloxy group or a hydroxy group represented by R^{2c} include 2,2-dimethylpropyl, 3-hydroxy-2,2-dimethylpropyl, 3-hydroxy-2-hydroxymethyl-2-methylpropyl, 3-acetoxy-2,2-dimethylpropyl, 3-acetoxy-2-hydroxymethyl-2-methylpropyl and 3-acetoxy-2-acetoxymethyl-2-methylpropyl. Among these, 2,2-dimethylpropyl, 3-hydroxy-2,2-dimethylpropyl and 3-acetoxy-2,2-dimethylpropyl are particularly preferred.

In addition, as R^{2c}, a C₃₋₆ alkyl group having an alkanoyloxy group and/or hydroxy group is preferred.

Examples of the lower alkyl group represented by R^{3c} include a C₁₋₆ alkyl group such as methyl, ethyl, n-propyl, isopropyl, n-butyl, tert-butyl, pentyl, hexyl. Among these, a C₁₋₃ alkyl group is preferred. In view of the pharmacological activity, methyl group is particularly preferred as R^{3c}.

Examples of the halogen atom represented by W include chlorine, fluorine, bromine and iodine atom. Among these, chlorine atom is preferred.

The present invention includes the compound represented by the formula (Ic) in the form of either free or a pharmacologically acceptable salt thereof. As such salt, when the compound represented by the formula (Ic) has an acidic group such as carboxyl group, it may form a salt with an inorganic base (e.g., alkali metals such as sodium and potassium, alkaline earth metals such as calcium and magnesium, transition metals such as zinc, iron and copper, etc.) or an organic base (e.g., organic amines such as trimethylamine, triethylamine, pyridine, picoline, ethanolamine, diethanolamine, triethanolamine, dicyclohexylamine and N,N′-dibenzylethylenediamine, and basic amino acids such as arginine, lysine and ornithine, etc.).

In case where the compound represented by the formula (Ic) of the present invention has a basic group such as amino group, it may form a salt with inorganic acids or organic acids (e.g., hydrochloric acid, nitric acid, sulfuric acid, phosphoric acid, carbonic acid, bicarbonic acid, formic acid, acetic acid, propionic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, etc.), and acidic amino acid such as aspartic acid, glutamic acid, and the like.

The compound represented by the formula (Ic) or a salt thereof has asymmetric carbon atoms at 3- and 5-position, but it may be in a mixture of the stereoisomers, and the isomers may also be separated by conventional means. The trans isomer wherein the substituents on 3- and 5-positions are directed in the opposite direction relative to the plane of the 7-membered ring is preferred, and in particular, the isomer wherein the absolute configuration at 3-position is R-configuration and the absolute configuration at 5-position is S-configuration is preferred. In addition, it may be a racemic compound or an optically active isomer. The optically active isomer can be separated from the racemic compound by a known optical resolution means.

As the compound represented by the formula (Ic) of the present invention or a salt thereof, the following compounds are preferred specifically.
N-propanesulfonyl-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetamide or a salt thereof;
(2R)-2-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminopropionic acid or a salt thereof;
3-[3-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminophenyl]propionic acid or a salt thereof;
4-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminobutanoic acid or a salt thereof;
trans-4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]-aminomethyl-1-cyclohexane carboxylic acid or a salt thereof;
trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-aminomethyl-1-cyclohexanecarboxylic acid or a salt thereof;
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorophenyl]propionic acid or a salt thereof;
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methylphenyl]propionic acid or a salt thereof;
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylphenyl]propionic acid or a salt thereof;
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid or a salt thereof;
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenyl]propionic acid or a salt thereof;
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]propionic acid or a salt thereof;
2-[2-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]ethyl]furan-3-carboxylic acid or a salt thereof;
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]propionic acid or a salt thereof;
3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid or a salt thereof;
4-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]butanoic acid or a salt thereof;
5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]pentanoic acid or a salt thereof;
5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]pentanoic acid or a salt thereof.

The compound represented by the above-mentioned formula (Ic) or a salt thereof can be produced, for example, according to a method disclosed in EP A 567,026, WO95/21834 (international application based on Japanese Patent Application No. 6-15531), EP A 645,377 (application based on Japanese Patent Application No. 6-229159), EP A 645,378 (application based on Japanese Patent Application No. 6-229160), WO01/98282 (international application based on Japanese Patent Application No. 2000-190253) and the like, or analogous methods thereto.

As raw materials of the compound represented by the formula (I) of the present invention, the same salts as those mentioned above can be used, but they are not particularly limited as long as they do not interfere with the reaction.

As the compound represented by the formula (I) or salts thereof, the following compound represented by formula (Id) is preferred.

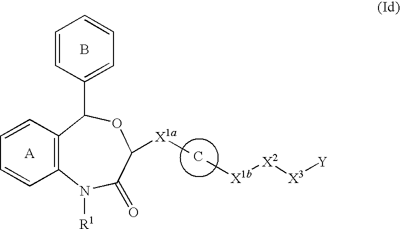

In the formula (Id), ring A and ring B each represent an optionally substituted benzene ring, ring C represents an optionally further substituted aromatic ring, R¹ represents a lower alkyl group optionally substituted with an optionally substituted hydroxyl group, X^{1a} represents a bond or optionally substituted lower alkylene, X^{1b} represents a bond or optionally substituted lower alkylene, X² represents a bond, —O— or —S—, X³ represents a bond or an optionally substituted divalent hydrocarbon group, and Y represents an optionally esterified or amidated carboxyl group.

Preferable examples of the compound represented by the formula (Id) include:
5-(3-{[(3R,5S)-7-chloro-5-(2,3-dimethoxyphenyl)-1-(2,2-dimethylpropyl)-2-oxo-1,2,3,5-tetrahydro-4,1-benzoxazepin-3-yl]methyl}-1,2,4-oxadiazol-5-yl)pentanoic acid or a salt thereof;
5-(3-{[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]methyl}-1,2,4-oxadiazol-5-yl)pentanoic acid or a salt thereof;
5-(3-{[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]methyl}-1,2,4-oxadiazol-5-yl)pentanoic acid or a salt thereof; and
(4-{[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl}phenyl)acetic acid or a salt thereof.

The “CRP-lowering” in the present invention refers to, with respect to the symptom showing an elevated in vivo (e.g., in blood) CRP level caused by various factors (in the present specification, such status of the elevated blood CRP levels (e.g. not less than 0.3 mg/dL) is referred to as “hyper C-reactive proteinemia”), an action of lowering the level to less than that of before-administration and bringing it close to normal value, that is, clinically, showing a therapeutic or preventive effect for various diseases associated with an elevation of CRP level.

According to the present invention, since SSI compound has a blood CRP-lowering activity, it is useful for treating and preventing diseases associated with an elevation of CRP level, for example, inflammatory disease, cancer, and the like. Herein, the “inflammatory disease” should be interpreted in the broadest sense of the word, including all disorders (disease or pathology) associated with inflammation (including the case caused by inflammation and the case developing inflammation as a result), and examples thereof include an infectious inflammation (e.g., inflammation caused by infection with bacteria, virus, fungus, protozoan, or other parasites), an allergic complication of infection (e.g., rheumatic fever, glomerulonephritis, erythema nodosum leprosum (ENL), etc.), chronic inflammatory disease (e.g., rheumatoid arthritis, juvenile arthritis, ankylosing spondylitis, psoriatic arthritis, systemic vasculitis, polymyalgia rheumatica (PMR), Reiter syndrome, Crohn's disease, familial Mediterranean fever, etc.), necrotic inflammation (e.g., acute pancreatitis, etc.), traumatic inflammation (e.g., inflammation caused by burn, operation, physical or chemical injury, or fracture etc.), angiopathy (e.g., coronary disease, aneurysm, arteriosclerosis such as atherosclerosis (including atherosclerosis by cardiac transplantation), cardiac infarction, embolism, peripheral arterial obstruction, restenosis after PTCA, apoplexy, thrombosis (including venous thrombosis), angina pectoris (including unstable angina pectoris), calcification (including vascular calcification and valvular calcification), Kawasaki's disease, other inflammatory angiopathy, etc.). In addition, examples of cancer include malignant lymphoma (Hodgkin's disease, non-Hodgkin lymphoma), ovary cancer, kidney cancer, pancreas cancer, cancer of gastrointestinal tract (including esophagus, stomach, colon, rectum), multiple myeloma, melanoma, malignant fibrous histiocytoma, and the like, but are not limited thereto. Namely, SSI compound can be used effectively as a preventive and therapeutic agent for rheumatism, cancer, thrombogenic disease and broad inflammatory diseases based on the CRP lowering activity which has been found newly in the present invention, as well as conventional known use such as preventive and therapeutic agent for hyperlipidemia, arteriosclerosis (atherosclerosis) and the like, triglyceride lowering agent, lipid lowering agent, high density lipoprotein-cholesterol elevating agent, antimycotic agent, skeletal muscle protecting agent and the like. Furthermore, it is useful as a preventive and therapeutic agent for various organ disorders due to angiopathy caused by arteriosclerosis (atherosclerosis) and vasculitis.

In addition, CRP is said to be arteriosclerosis (atherosclerosis) risk factor (initiation factor) independent of blood cholesterol, and elevation of blood CRP level (hyper-CRPemia) can be deemed to be one of diseases different from hyperlipidemia. Thus, SSI compound is useful for prevention and treatment of hyper-CRPemia.

As mentioned above, since CRP is shown to exert as an exacerbation factor for the progression of arteriosclerosis (atherosclerosis) lesion and plaque rupture, SSI compound is particularly useful for inhibition of progression of arteriosclerosis (atherosclerosis) plaque and/or stabilization thereof.

By the way, in the patients of ischemic heart disease who develop cardiac infarction and the like, there are a considerable number of patients having a high CRP value, although the blood cholesterol level is within a range of normal level. The prevention, inhibition of progression and treatment for ischemic heart disease can be achieved based on the CRP lowering activity of SSI compound by administering the SSI compound to patients having ischemic heart disease or patients having high developing risk thereof. The pharmaceutical use of the SSI compound to such certain patients has become to be applicable for the first time by the new knowledge of the present invention that SSI compound has a CRP lowering activity.

In addition, for example, HMG-CoA reductase inhibitor is known as a drug having CRP lowering activity other than SSI compound, but some muscle symptoms such as rhabdomyolysis and the like may be observed as a side effect. The SSI compound has no side effect like this, and has an advantage of being safely administered.

The SSI compound used in the present invention is low toxic (for example, more excellent as a drug in view of acute toxicity, chronic toxicity, generative toxicity, genetic toxicity, cardiac toxicity, drug interaction, carcinogenicity and the like). Accordingly, the compound can be used safely as a medicine as it is, or as a pharmaceutical composition by mixing with a pharmaceutically acceptable carrier known per se to a mammal (for example, human, monkey, cattle, horse, pig, mouse, rat, hamster, rabbit, cat, dog, sheep, goat and the like).

In the agent of the present invention, the compound having inhibitory activity against squalene synthase or a salt thereof, or a prodrug thereof (hereinafter, also referred to as an “SSI compound or a prodrug thereof”) which is an active ingredient, can be administered as bulk powder, or usually in the form of a pharmaceutical composition or preparation which is prepared by a conventional method using carriers for formulation in suitable amount, which are suitably selected from, for example, an excipient (e.g., calcium carbonate, kaolin, sodium hydrogen carbonate, lactose, starches, crystalline cellulose, talc, granulated sugar, porous substances, etc.), a binder (e.g., dextrin, gums, alcoholized starch, gelatin, hydroxypropylcellulose, hydroxypropylmethylcellulose, pullulan, etc.), a disintegrating agent (e.g., carboxymethylcellulose calcium, sodium croscarmellose, crospovidone, low-substituted hydroxypropylcellulose, partially pregelatinated starch, etc.), a lubricant (e.g., magnesium stearate, calcium stearate, talc, starch, sodium benzoate, etc.), a colorant (e.g., tar dye, caramel, iron sesquioxide, titanium oxide, riboflavins, etc.), a flavoring substance (e.g., sweeters, flavors, etc.), a stabilizer (e.g., sodium sulphite, etc.), a preservative (e.g., parabens, sorbic acid, etc.) and the like. The agent of the present invention including the above-mentioned preparations contains suitably the SSI compound or prodrug thereof in an effective amount for treating and preventing the diseases. The content of the SSI compound or prodrug thereof in the preparation of the present invention is usually 0.1 to 100% by weight based on the total preparation. Furthermore, the preparation used in the present invention may contain other drug ingredients as active ingredients, in addition to the SSI compound or prodrug thereof. Such ingredient is not particularly limited as long as the object of the present invention is achieved, and can be used in a suitable mixing ratio. Specific examples of the preparations include tablets (including sugar-coated tablets and film-coated tablets), pills, capsules, granules, fine-granules, powders, syrup, emulsion, suspension, injectable preparation, sustained release injectable preparation, inhalant, ointment, etc. These preparations can be prepared according to a conventional method (for example, a method described in Japanese Pharmacopoeia).

Specifically, tablets can be prepared by granulating suitably the SSI compound or a prodrug thereof as it is, or with an excipient, a binder, a disintegrating agent or other suitable additives, followed by adding a lubricant and the like and then compressing and molding the mixture, or by compressing and molding directly the SSI compound or a prodrug thereof as it is, or a homogenous mixture thereof with an excipient, a binder, a disintegrating agent or other suitable additives. Alternatively, tablets can be prepared by compressing and molding granules previously prepared as they are, or a homogenous mixture thereof with suitable additives. Furthermore, a colorant, a flavoring substance and the like can be added to the present preparation, if necessary. Furthermore, the present preparation can be coated with a suitable coating agent. The injectable preparation can be prepared by dissolving, suspending or emulsifying a certain amount of the SSI compound or a prodrug thereof in an aqueous solvent such as water for injection, physiological saline, Ringer solution and the like, or in a non-aqueous solvent such as, usually, vegetable oil to prepare a certain amount of the injectable solution, or, by sealing a certain amount of the SSI compound or a prodrug thereof in a vessel for injection.

Examples of carriers for oral preparations include substances conventionally used in the field of formulation such as starch, mannitol, crystalline cellulose and carboxymethylcellulose sodium. Examples of carriers for injection include distilled water, physiological saline, glucose solution, infusion solution and the like. Also, other additives which are used for formulation in general can be suitably added.

Furthermore, the preparation of the present invention can be used as a sustained-release preparation. The sustained-release preparation can be administered as microcapsules (for example, microsphere/microcapsules, micro-particles and the like) as they are, which are prepared by a method such as drying-in-water method (o/w method, w/o/w method and the like), phase separation method, spray drying method or a similar method thereto, or as other various preparations formulated starting from a pharmaceutical composition in the form of microcapsules, or spheres, needles, pellets, film or cream. Examples of the dosage form include parenteral preparations (for example, an injectable preparation or an implant for intramuscular, subcutaneous, organ; an intramucosal preparation for nasal cavity, rectum, uterus and the like), oral preparations (for example, hard capsules, soft capsules, granules, powders, suspension and the like) and the like.

When the sustained-release preparation is an injectable preparation, it is prepared as an aqueous suspension by dispersing microcapsules with a dispersant (for example, a surfactant such as Tween 80 and HCO-60; polysaccharide such as carboxymethylcellulose, sodium alginate and sodium hyaluronate; protamine sulfate, polyethylene glycol and the like), a preservative (for example, methylparaben, propylparaben and the like), an isotonic agent (for example, sodium chloride, mannitol, sorbitol, glucose and the like), a local anesthetic (for example, xylocaine hydrochloride, chlorobutanol and the like), or as an oily suspension by dispersing the microcapsules in a vegetable oil (for example, sesame oil, corn oil and the like) or in a mixture thereof with phospholipid (for example, lecithin and the like), or with middle chain triglyceride (for example, Miglyol 812 and the like).

When the sustained-release preparation is microcapsules, the mean particle diameter is about 0.1 to about 300 μm, preferably, about 1 to about 150 μm, more preferably about 2 to about 100 μm.

A method of preparing the microcapsules as aseptic preparations includes a method wherein all the processes are conducted under aseptic conditions, sterilization with gamma rays, addition of an antiseptic and the like, which are not particularly limited thereto.

Dose of the agent of the present invention is varied depending on an administration route, symptoms, the age or body weight of the patient and the like. For example, when orally administered to an adult patient as a preventing and/or treating agent for arteriosclerosis (atherosclerosis), it is preferable to administer 1 to 400 mg/day, preferably 6 to 1.20 mg/day as the SSI compound once or in several times a day. The administration route may be oral or parenteral.

Furthermore, the dose of the sustained-release preparation as an example of the agent of the present invention is varied depending on the duration of release as well as the administration route, symptoms, the age or weight of the patient and the like. However, it is not particularly limited if it is an amount to maintain the effective concentration of the active ingredient in the body and the number of administration can be suitably selected depending on the situation, for example, once a day to once 3 days, or once a week to once 3 months.

SSI compound can be used together with other drugs. Therefore, the present invention provides also a concomitant drug comprising a combination of SSI compound and other drugs.

Examples of the drugs which can be used together with SSI compound as the concomitant drug of the present invention (hereinafter, sometimes abbreviated as combined drug) include a drug having a CRP lowering activity other than SSI compound, or a drug showing a preventive and/or therapeutic effect for any of the above-mentioned various diseases associated with an elevation of CRP level. Examples of the drug having a CRP lowering activity other than SSI compound include HMG-CoA reductase inhibitor (e.g., refer to U.S. Pat. No. 4,444,784), the compound disclosed in US-A 2003/0171251 as general formula (I), benzofuran compound disclosed in U.S. Pat. No. 6,653,346 as general formula (I), and the like, but not limited thereto. Although there are rare occasions that an HMG-CoA reductase inhibitor shows muscle symptoms such as rhabdomyolysis etc. as a side effect, the SSI compound of the present invention has also skeletal muscle protecting action, therefore it is supposed that by the concomitant use, not only the dose of HMG-CoA reductase inhibitor is simply decreased, but also the development of muscle symptoms due to HMG-CoA reductase inhibitor can actively be inhibited.

On the other hand, examples of the drug showing a preventive and/or therapeutic effect for the diseases associated with an elevation of CRP level include an anti-inflammatory drug, antirheumatic drug, antibacterial drug, antimycotic agent, antiviral drug, antiallergic drug, anti-angiopathic drug, anticancer agent, and the like, but are not limited thereto.

More specifically, examples of the anti-inflammatory drug include a non-steroidal anti-inflammatory/analgesic drug which is cyclooxygenase (COX) inhibitor (e.g., various salicylic acid drugs such as aspirin; anthranilic acid drugs such as mefenamic acid and flufenamic acid; indole acetic acid drugs such as indometacin, sulindac and acemetacin; phenylacetic acid drugs such as diclofenac and fenbufen; propionic acid drugs such as ibuprofen, ketoprofen, loxoprofen naproxen and tiaprofen; oxicam drugs such as piroxicam, tenoxicam and ampiroxicam; pyrazolone drugs such as ketophenylbutazone; and the like), anti-cytokine drugs (e.g., anti-cytokine antibody such as anti-TNF-α antibody and anti-IL-6 antibody, antisense oligonucleotide of cytokine gene, cytokine binding protein, and the like) and the like.

Examples of the antirheumatic drug include aurate preparations such as sodium aurothiomalate and auranofin; penicillamine drugs such as bucillamine and penicillamine; lobenzarit drugs such as lobenzarit disodium; actarit, salazosulfapyridine, methotrexate, mizoribine, cyclosporin, azathioprine, cyclophosphamide, prednisolone farnesylate, and the like.

Examples of the antibacterial drug include penicillin antibiotics (e.g., amoxicillin, ampicillin, bacampicillin, etc.), cephem antibiotics (e.g., cefalexin, cefaclor, cefdinir, cefteram pivoxil, cefixime, cefotiam hydrochloride, etc.), macrolide antibiotics (e.g., erythromycin, clarithromycin, roxithromycin, josamycin, etc.), tetracycline antibiotics (e.g., minocycline, doxycycline, demeclocycline, etc.), fosfomycin antibiotics (e.g., fosfomycin,), aminoglycoside antibiotics (e.g., kanamycin, etc.), new quinolone antibacterial drug (e.g., levofloxacin, ofloxacin, norfloxacin, tosufloxacin, etc.), and the like, and examples of the antimycotic agent include polyene antifungal drugs (e.g., trichomycin, amphotericin B, nystatin, etc.), imidazole antifungal drugs (e.g., econazole, miconazole, clotrimazole, etc), triazole antifungal drugs (e.g., fluconazole, itoraconazole, etc.), allylamine antifungal drugs (e.g., butenafine, terbinafine hydrocloride, etc.), flucytosine (5-FC) antifungal drugs (e.g., flucytosine, etc.), and the like. Examples of the antiviral drug include nucleic acid synthesis inhibitory antiviral drugs (e.g., acyclovir, ganciclovir, vidarabine, foscarnet, zidovudine, lamivudine, didanosine, etc.), intracellular invasion inhibitory antiviral drugs (e.g., amantadine, zanamivir, oseltamivir etc.), host phylaxis ability enhancing antiviral drugs (e.g., interferon, inosine pranobex, etc.), and the like.

Examples of the antiallergic drug include antihistaminic antiallergic drugs (e.g., ketotifen, azelastine, oxatomide, mequitazine, epinastine hydrochloride, terfenadine, etc.), non-antihistaminic antiallergic drugs (e.g., ozagrel hydrochloride, sodium cromoglicate, tranilast, repirinast, amlexanox, etc.), and the like.

Examples of the anti-angiopathic drug include cilostazol, abciximab, and the like.

Examples of the anticancer agent include molecule targeting drugs (e.g., trastuzumab, rituximab, imatinib, gefitinib, etc.), alkylating drugs (e.g., cyclophosphamide, cisplatin, etc.), antimetabolite (e.g., methotrexate, 6-mercaptopurine, 5-FU, etc.), antibiotics (e.g., bleomycin, adriamycin, actinomycin D, etc.), plant alkaloids (e.g., vincristine, vinblastine, paclitaxel, etc.), hormones (e.g., prednisolone, tamoxifen, etc.), and the like.

The administration mode of the SSI compound and the combined drug to be used in the present invention is not particularly limited, and the SSI compound and the combined drug may be combined before administration. Examples of such administration mode include the following methods:

(1) administration of a single preparation prepared by formulating the SSI compound and the combined drug simultaneously, (2) simultaneous administration of two kinds of preparations obtained by formulating the SSI compound and the combined drug separately, via a single administration route, (3) separate administration at an interval of two kinds of preparations obtained by formulating the SSI compound and the combined drug separately, via a single administration route, (4) simultaneous administration of two kinds of preparations obtained by formulating the SSI compound and the combined drug separately, via different administration routes, (5) separate administration at an interval of two kinds of preparations obtained by formulating the SSI compound and the combined drug separately, via different administration routes (e.g., administration of the SSI compound followed by the combined drug, or administration in the reverse order). Dose of the combined drug can be appropriately selected based on the dose which is clinically used. The compounding ratio of the SSI compound and the combined drug can be appropriately selected depending on the kind of combined drug, administration subject, administration route, target diseases, symptoms, combinations thereof, etc. For example, when HMG-CoA reductase inhibitor is administered as a combined drug to human, the SSI compound may be used in an amount of 0.01 to 100 parts by weight relative to 1 part by weight of the HMG-CoA reductase inhibitor.

As mentioned above, since compounds having CRP lowering activity exhibit a preventive and/or therapeutic effect for traumatic inflammation and tissue damage (necrosis), the SSI compound can be used for preventing damage and aging of skin such as sunscreen, skin-whitening and wrinkle reduction by formulating the SSI compound as it is or a mixture thereof with appropriate additives such as an excipient etc., into non-medical external preparation (quasi drug, cosmetics etc.; hereinafter, sometimes abbreviated simply as “external preparation of the present invention”).

The external preparation of the present invention can be in the form of aqueous solution, oil, other solutions, emulsion, cream, gel, suspension, microcapsules, powder, granules, and the like. By preparing these forms with a method known per se, they can be applied, plastered or sprayed to the body as lotion, emulsion, cream, ointment, plaster, cataplasm, aerosol or the like.

In addition to conventionally used excipients, flavors and the like, lipids, surfactants, antiseptics, metal ion sealing agent, water-soluble polymers, thickening agent, powder ingredients, ultraviolet protectors, moisturizing agent, other medicinally active ingredients, antioxidants, pH adjusters, cleansing agent, drying agent, emulsifying agent and the like can be suitably compounded into the external preparation of the present invention.

Examples of the lipids include liquid lipids (e.g., avocado oil, camellia oil etc.), solid lipids (e.g., cacao oil, coconut oil, horse lipid, hydrogenated coconut oil etc.), waxes (e.g., beeswax, canderilla wax, cotton wax, carnauba wax etc.), hydrocarbon oils (e.g., liquid paraffin, paraffin etc.), higher fatty acids (e.g., lauric acid, myristic acid, palmitic acid, stearic acid, oleic acid etc.), higher alcohols (e.g., linear alcohols such as lauryl alcohol, branched alcohols such as monostearyl glycerin ether (batyl alcohol) etc.), synthetic ester oils (e.g., isopropyl myristate, cetyl octanoate etc.), silicons (e.g., chain polysiloxanes such as dimethyl polysiloxane, cyclic polysiloxanes such as decamethyl polysiloxane, silicon resin having three-dimensional network, silicone gum etc.).

Examples of the surfactants include anion surfactants (e.g., sodium laurate, sodium laurylsulfate, sodium lauroylsarcosine, hydrogenated palm oil fatty acid glycerosulfuric acid sodium salt, Turkey red oil etc.), cation surfactants (e.g., stearyltrimethylammonium chloride, polyamine fatty acid derivatives, amyl alcohol fatty acid derivatives, benzalkonium chloride etc.), amphoteric surfactants (e.g., imidazoline ampholytic surfactants such as 2-undecyl-N,N,N-(hydroxyethylcarboxymethyl)-2-imidazoline sodium, betaine surfactants such as 2-heptadecyl-N-carboxymethyl-N-hydroxyethylimidazolinium betaine etc.), non-ionic surfactants (e.g., sorbitan fatty acid esters such as sorbitan monooleate, glycerine polyglycerol fatty acids such as glyceryl monocottonseed oil fatty acid, propylene glycol fatty acid esters such as propylene glycol monostearate, hydrogenated caster oil derivative, polyoxyethylene methylpolysiloxane copolymer etc.).

Examples of the antiseptics include methylparaben, ethylparaben, butylparaben and the like.

Examples of the metal ion sealing agent include edetic acid sodium salt, EDTA and the like.

Examples of the water-soluble polymers include natural polymers (e.g., vegetable polymers such as gum arabic, tragacanth gum, starch and glycyrrhizic acid; micobial polymers such as xanthan gum, dextrans and pullulan; animal polymers such as collagen, casein, albumin and gelatin; etc.), semi-synthetic polymers (e.g., starch polymers such as dextrin and methylhydroxypropylstarch; cellulose polymers such as methylcellulose, nitrocellulose, methylhydroxypropylcellulose, hydroxypropylcellulose, sodium carboxymethylcellulose (CMC) and crystalline cellulose; arginic acid polymers such as sodium arginate and propylene glycol arginate ester etc.), synthetic polymers (e.g., vinyl polymers such as polyvinyl alcohol, polyvinylpyrrolidone and carboxyvinyl polymer; polyoxyethylene polymers such as polyethylene glycol 2000, 4000 and 6000; polyoxyethylene polyoxypropylene copolymers; acrylic polymers such as sodium polyacrylate and polyacrylamide; polyethyleneimine, cation polymer, etc.) and inorganic polymers (e.g., bentonite, magnesium alminium silicate, anhydrous silicic acid etc.).

Examples of the powder ingredients include talc, kaolin, mica, magnesium carbonate, alminium silicate, tungstic acid metal salts, silica, zeolite, barium sulfate, calcined calcium sulfate (calcinedgypsum), calcium phosphate, hydroxyapatite, metal soap (zinc myristate, calcium palmitate, alminium stearate), inorganic powder such as boron nitride, polyamide resin powder (nylon powder), resin powder of copolymer of styrene and acrylic acid, organic powder such as cellulose powder, inorganic white pigment such as titanium dioxide and zinc oxide, inorganic red pigment such as iron oxide (bengala) and iron titanate, inorganic brown pigment such as γ-iron oxide, inorganic yellow pigment such as yellow iron oxide, inorganic black pigment such as black iron oxide, inorganic purple pigment such as mango violet, inorganic green pigment such as chromic oxide, inorganic blue pigment such as ultramarine blue, pearl pigment such as titanium oxide-coated mica, metal powder pigment such as aluminium powder, organic pigment such as zirconium, barium or aluminium lake such as Red No. 201, Red No. 202, Orange No. 203, Orange No. 204, Yellow No. 205, Yellow No. 401, Blue No. 404, Red No. 3, Red No. 104, Orange No. 205, Yellow No. 4, Yellow No. 5, Green No. 3 and Blue No. 1, natural pigment such as chlorophyll and β-carotene, and coloring agent such as titanium yellow and safflower red, and the like.

Examples of the ultraviolet protectors include ultraviolet absorbers which absorb ultraviolet rays chemically (e.g., absorbers for long-wave ultraviolet light (UVA) such as 4-methoxy-4′-tert-butylbenzoylmethane, 2-hydroxy-4-methoxybenzophenone and 2-hydroxy-4-methoxybenzophenone derivatives; absorbers for middle wavelength ultraviolet rays (UVB) such as benzoic acid ultraviolet absorbers such as para-aminobenzoic acid (PABA), salicylic acid ultraviolet absorbers such as dipropylene glycol salicylate, cinnamic acid ultraviolet absorbers such as octyl cinnamate, and camphor derivatives such as 3-(4′-methylbenzylidene)-d,1-camphor), and ultraviolet screening agents which scatter or reflect ultraviolet rays physically (e.g., titanium oxide, talc, carmine, bentonite, kaolin, zinc oxide etc.).

Examples of the moisturizing agent include polyethylene glycol, propylene glycol, glycerin, xylitol, sorbitol, maltitol, chondroitin sulfate, hyaluronic acid, mucoitinsulfuric acid, atelocollagen, cholesteryl-12-hydroxystearate, sodium lactate, bile acid salt, extract of Rosa roxburghii, extract of Achillea millefolium, and the like.

Examples of the other medicinally active ingredients include skin-whitening agents such as arbutin, vitamin C and derivatives thereof, kojic acid, extract of placenta, glutathione and extract of saxifrage; antiinflammatory agents such as glycyrrhizic acid derivatives, glycyrrhetic acid derivatives, salicylic acid derivatives and hinokitiol; activating agents such as royal jelly, photosensitive element and cholesterol derivatives; blood circulation accelerator such as nonylic vanillylamide, benzyl nicotinate capsaicine, caffeine, tannic acid, tocopherol nicotinate and acetylcholine; anti-seborrhea agent such as sulfur and thianthol; for various purposes, extract of phellodendron bark, extract of coptis rhizome, extract of lithospermum root, extract of peony, extract of swertia, extract of sage, extract of eriobotryae, extract of ginseng, extract of aloe, extract of luffah, extract of lily, extract of saffron, extract of guttiferae, extract of rosemary, extract of garlic; and vitamins such as vitamin As, vitamin B₂s, vitamin Cs, pantothenic acids, nicotinic acids, vitamin Es, vitamin P and biotin.

The content of the SSI compound to be contained in the external preparation of the present invention is not particularly limited, as long as it is in a range of enough amounts to exert a preventive and/or therapeutic effect for tissue damage and have no adverse effects on living body. For example, it can be compounded in a rage of about 0.01 to about 20% by weight.

### EXAMPLES

Hereinafter, test results showing the pharmacological effects of the agent of the present invention are described. This is just an example, and the present invention is not limited by the Examples.

### Test Compound 1:

### N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid

Test compound 1 is a compound described in Example 36 of JP-A No. 09-136880, and can be synthesized by the method described in this publication and the like.

### Test Example 1

### Plasma CRP Lowering Effect

Method:

To 2 month-old male WHHL rat (11 per group) was orally administered with a mixed food a vehicle or Test compound 1 at a dose of 100 and 200 mg/kg for 28 days. Before administration and after 28 days administration, plasma CRP level was determined using CRPα Test Wako (Wako Pure Chemical Industries, Ltd.) with Hitachi, Ltd. Autoanalyzer 7070 (Table 1).

Results:

TABLE 1

| | | Plasma CRP | Plasma CRP |
|---|---|---|---|
| | | level (mg/dL) | level (mg/dL) |
| | Dose | Value of before | After 28 days |
| Treatment | (mg/kg) | administration | administration |
| Vehicle | 0 | 3.8 ± 0.5 | 5.0 ± 0.7 |
| Test | 100 | 4.4 ± 0.4 | 3.2 ± 0.6 |
| compound 1 | | | |
| Test | 200 | 4.4 ± 0.6 | 2.1 ± 0.2* |
| compound 1 | | | |

Data represent Mean ± SE (N = 11).
*P < 0.025 vs. Control (one-tailed Williams' test)

Results of Table 1 showed that the SSI compound decreased the CRP concentration in the plasma.

### PREPARATION EXAMPLES

The CRP lowering agent of the present invention can be produced, for example, by the following prescription.

In addition, for ingredients other than the active ingredients (additives) described in the following prescription, products described in Japanese Pharmacopoeia, Japanese Pharmaceutical Codex, or The Japanese Standards of Drug Additives can be used.

1. Capsule

| | | |
|---|---|---|
| (1) N-[[(3R,5S)-1-(3-acetoxy-2,2-dimethylpropyl)-7-chloro- | 10 | mg |
| 5-(2,3-dimethoxyphenyl)-2-oxo-1,2,3,5-tetrahydro-4,1- | | |
| benzoxazepin-3-yl]acetyl]piperidine-4-acetic acid | | |
| (2) Lactose | 90 | mg |
| (3) Microcrystalline cellulose | 70 | mg |
| (4) Magnesium stearate | 10 | mg |
| 1 capsule | 180 | mg |

(1), (2) and (3) and the half of (4) are kneaded and then granulated. To this is added the remaining (4), and the whole is sealed into a gelatin capsule.

2. Tablet

| | | |
|---|---|---|
| (1) N-[[(3R,5S)-1-(3-acetoxy-2,2-dimethylpropyl)-7-chloro- | 10 | mg |
| 5-(2,3-dimethoxyphenyl)-2-oxo-1,2,3,5-tetrahydro-4,1- | | |
| benzoxazepin-3-yl]acetyl]piperidine-4-acetic acid | | |
| (2) Lactose | 35 | mg |
| (3) Corn starch | 150 | mg |
| (4) Microcrystalline cellulose | 30 | mg |
| (5) Magnesium stearate | 5 | mg |
| 1 tablet | 230 | mg |

(1), (2), (3), ⅔ of (4) and the half of (5) are kneaded and then granulated. The remaining (4) and (5) are added to the granules, and compressed and molded into tablets.

3. Injection

| | | |
|---|---|---|
| (1) N-[[(3R,5S)-1-(3-acetoxy-2,2-dimethylpropyl)-7-chloro- | 10 | mg |
| 5-(2,3-dimethoxyphenyl)-2-oxo-1,2,3,5-tetrahydro-4,1- | | |
| benzoxazepin-3-yl]acetyl]piperidine-4-acetic acid | | |
| (2) Inositol | 100 | mg |
| (3) Benzyl alcohol | 20 | mg |
| 1 Ampoule | 130 | mg |

(1) , (2) and (3) are dissolved in distilled injection solvent so as to be a total of 2 ml, which is sealed into an ampoule. All steps are conducted under sterilized conditions.

### INDUSTRIAL APPLICABILITY

Since the compound having a squalene synthase inhibitory activity to be used in the present invention is low toxic and has an excellent CRP lowering activity, a safe and an effective preventive and/or therapeutic agent for various diseases involved in elevation of CRP level, in particular, inflammatory disease and cancer can be provided by the present invention.

1. A CRP lowering agent comprising a compound having a squalene synthase inhibitory activity or a salt thereof, or a prodrug thereof.

2. The agent according to claim 1, which is a preventive and/or therapeutic agent for inflammatory diseases.

3. The agent according to claim 1, which is a preventive and/or therapeutic agent for hyper C-reactive proteinemia.

4. The agent according to claim 1, which is an inhibitor of development of arteriosclerotic (atherosclerotic) plaque or a stabilizer thereof.

5. The agent according to claim 1, wherein the compound having a squalene synthase inhibitory activity is a compound represented by the formula: 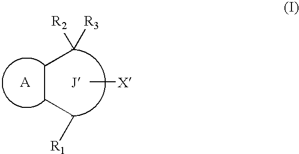
wherein, R₁ represents a hydrogen atom or an optionally substituted hydrocarbon group, R₂ and R₃ are the same or different and represent a hydrogen atom, an optionally substituted hydrocarbon group or an optionally substituted heterocyclic group, X′ represents a group comprising an optionally esterified carboxyl group, an optionally substituted carbamoyl group, an optionally substituted hydroxy group, an optionally substituted amino group or an optionally substituted heterocyclic residue having a hydrogen atom which can be deprotonated, Ring A represents an optionally substituted benzene ring or an optionally substituted heterocyclic ring, Ring J′ represents a 7- to 8-membered heterocyclic ring containing 3 or less hetero atoms as ring constituent atoms, and Ring J′ may further have a substituent in addition to R₁, R₂, R₃, and X′.

6. The agent according to claim 1, wherein the compound having a squalene synthase inhibitory activity is a compound represented by the formula: 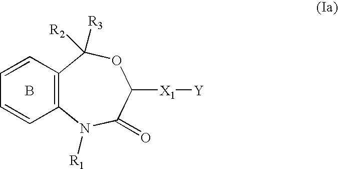
wherein, R₁ represents a hydrogen atom or an optionally substituted hydrocarbon group, R₂ and R₃ are the same or different and represent a hydrogen atom, an optionally substituted hydrocarbon group or an optionally substituted heterocyclic group, X₁ represents a bond or a divalent atomic chain, Y represents an optionally esterified carboxyl group, an optionally substituted carbamoyl group, an optionally substituted hydroxy group, an optionally substituted amino group, or an optionally substituted heterocyclic residue having a hydrogen atom which can be deprotonated, Ring B represents an optionally substituted benzene ring.

7. The agent according to claim 1, wherein the compound having a squalene synthase inhibitory activity is a compound represented by the formula: 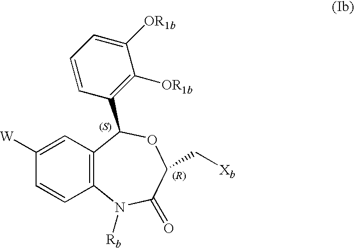
wherein, R_{b} represents a lower alkyl group optionally substituted with an optionally substituted hydroxy group X_{b} represents an optionally substituted carbamoyl group or an optionally substituted heterocyclic group having a hydrogen atom which can be deprotonated, R_{1b} represents a lower alkyl, and W represents a halogen atom.

8. The agent according to claim 7, wherein R_{b} is a C₁₋₆ alkyl which may have 1 to 3 substituents selected from hydroxy group, acetyloxy, propionyloxy, t-butoxycarbonyloxy, palmitoyloxy, dimethylaminoacetyloxy and 2-aminopropionyloxy.

9. The agent according to claim 7, wherein R_{1b} is methyl.

10. The agent according to claim 7, wherein W is chlorine atom.

11. The agent according to claim 7, wherein X_{b} is a group represented by the formula: 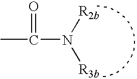
wherein R_{2b} and R_{3b} are each a hydrogen atom, an optionally substituted hydrocarbon group, an optionally substituted heterocyclic group or an acyl group, or R_{2b} and R_{3b} may be combined together with the adjacent nitrogen atom to form an optionally substituted 5- or 6-membered nitrogen-containing heterocyclic ring which may contain 1 to 3 hetero atoms selected from a nitrogen atom, a sulfur atom and an oxygen atom as ring constituent atoms.

12. The agent according to claim 7, wherein X_{b} is a group represented by the formula: 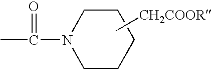
wherein R″ represents hydrogen atom or a C₁₋₄ alkyl.

13. The agent according to claim 1, wherein the compound having a squalene synthase inhibitory activity is N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-yl]acetyl]piperidine-4-acetic acid or N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepine-3-yl]acetyl]piperidine-4-acetic acid.

14. A method for preventing and/or treating a disease involved in elevation of CRP, which comprises inhibiting a squalene synthase in mammals.

15. A method for preventing and/or treating a disease involved in elevation of CRP, which comprises administering an effective amount of a compound having a squalene synthase inhibitory activity, or a prodrug thereof, or a salt thereof to a mammal.

16. Use of a compound having a squalene synthase inhibitory activity, or a prodrug thereof, or a salt thereof for producing a preventive and/or therapeutic agent of a disease involved in elevation of CRP.
